(19) United States
(12) Patent Application Publication
(10) Pub. No.: US 2025/0352097 A1
(43) Pub. Date: Nov. 20, 2025

(54) OPTICAL SPECTROSCOPY WITH CONTROLLED PATH LENGTH FOR NON-INVASIVE MEASUREMENT THROUGH SKIN

(71) Applicant: Si-Ware Systems, Cairo (EG)

(72) Inventors: Ahmed M. Othman, Cairo (EG); Bassem Mortada, Cairo (EG); Bassam Saadany, Cairo (EG); Diaa Khalil, Cairo (EG); Hisham Haddara, Nasr City (EG); Yasser M. Sabry, Cairo (EG); Shady Labib, Cairo (EG); Mohamed Ahmed Gaber, Cairo (EG)

(21) Appl. No.: 19/034,149

(22) Filed: Jan. 22, 2025

Related U.S. Application Data
(60) Provisional application No. 63/647,887, filed on May 15, 2024.
(60) Provisional application No. 63/651,576, filed on May 24, 2024.
(60) Provisional application No. 63/676,648, filed on Jul. 29, 2024.

(51) Int. Cl. A61B 5/1455; A61B 5/00; A61B 5/145; B60K 28/06

(52) U.S. Cl. CPC A61B 5/1455; A61B 5/14532; A61B 5/14546; A61B 5/6843; A61B 5/6893; A61B 2503/12; A61B 2562/028; B60K 28/063

(57) ABSTRACT

Aspects relate to mechanisms to control the effective optical path length through skin tissue for non-invasive optical spectroscopy measurements. An apparatus can include a path length control part configured to control the effective optical path length of diffusely scattered light transmitted through skin tissue to produce a target effective optical path length. The apparatus may further include a spectral sensor, a detector, and a light source configured to produce input light directed towards the path length control part or the spectral sensor. The detector is configured to obtain a spectrum of an analyte under test based on the diffusely scattered light. The spectral sensor is configured to either receive the input light, produce modulated light based on the input light, and direct the modulated light to the skin tissue, or to receive the diffusely scattered light from the skin tissue and obtain the spectrum using the detector.

### PRIORITY CLAIM

This application claims priority to and the benefit of Provisional Application No. 63/647,887, filed in the U.S. Patent and Trademark Office on May 15, 2024, Provisional Application No. 63/651,576, filed in the U.S. Patent and Trademark Office on May 24,2024, and Provisional Application No. 63/676,648, filed in the U.S. Patent and Trademark Office on Jul. 29, 2024, the entire contents of which are incorporated herein by reference as if fully set forth below in its entirety and for all applicable purposes.

### TECHNICAL FIELD

The technology discussed below relates generally to optical spectroscopy, and in particular to controlling the effective optical path length through skin.

### BACKGROUND

A spectrometer measures a single-beam spectrum (e.g., a power spectral density (PSD)). The intensity of the single-beam spectrum is proportional to the power of the radiation reaching the detector. In spectrometry absorbance of a sample is its fingerprint, which is used in spectral processing operations to enable material identification, along with quantitative and qualitative analysis. For non-invasive blood biochemistry measurements through skin, the skin has a high concentration of water (e.g., 60% to 80%), which has a strong absorption in the near and mid-infrared spectral regions. This causes the optical signal to face high attenuation. An optimal path length (e.g., optimal optical path length) is needed for efficient measurement through skin with a high signal-to-noise (SNR) ratio. Path lengths much greater or much smaller than the optimal path length causes a significant loss of measurement SNR.

The mid-infrared spectral range contains the fingerprint region for most material allowing the accurate analysis of blood biochemicals. However, the optimal path length for measurement in this range is in the 10 s of micrometers (μm), which is prohibitively short. The visible and infrared regions below the 1 μm wavelength range can allow a path length of 10 mm. However, the absorption features in this range for most biomarkers do not allow enough specificity.

The near infrared (NIR) wavelength range (e.g., 0.75 μm-1 μm) is useful for the detection of some physiological parameters, such as oxygen saturation measurements (e.g., pulse oximeters). Pulse oximeters are widely used in the non-invasive measurement of oxygen saturation using transmission or reflection spectroscopy through the skin, where the optimal path length is in the order of many centimeters. Thus, measuring in the transmission mode across a body part (e.g., a finger) is feasible. However, some other physiological parameters, such as glucose or blood alcohol, have weak absorption in this wavelength range and may be easier to detect in the short-wave infrared (SWIR) spectral range (e.g., 0.9 μm-2.5 μm). The SWIR range can allow the quantitative and qualitative detection of many biomarkers and analytes in blood. The optimal path length is about 0.25 mm to 2 mm, depending on the wavelength of interest for the biomarker. Thus, a mechanism for measurement through skin with a controlled optical path length close to the optimal value is needed.

Diffuse reflectance through skin can be used to achieve an effective optical path length near the optimal value. However, in diffuse reflectance, surface reflection from the skin can lead to a large undesired signal (stray light) that varies greatly with time, skin surface profile, and from subject to subject. In addition, diffuse reflectance suffers from large losses in optical signal due to dependence on random light path scattering through the skin until the signal is reflected back.

### SUMMARY

The following presents a summary of one or more aspects of the present disclosure, in order to provide a basic understanding of such aspects. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in a form as a prelude to the more detailed description that is presented later.

In an example, an apparatus configured for non-invasive optical spectroscopy is provided. The apparatus includes a path length control part configured to control an effective optical path length of diffusely scattered light non-invasively transmitted through skin tissue of a subject to produce a target effective optical path length through the skin tissue. The apparatus further includes a spectral sensor, a detector configured to obtain a spectrum of an analyte of the skin tissue under test based on the diffusely scattered light and a light source configured to produce input light and to direct the input light towards the path length control part or the spectral sensor. The spectral sensor is configured to either receive the input light produce modulated light based on the input light, and direct the modulated light to the path length control part to produce the diffusely scattered light from which the spectrum is obtained by the detector, or receive the diffusely scattered light from the path length control part and obtain the spectrum using the detector.

These and other aspects of the disclosure will become more fully understood upon a review of the detailed description, which follows. Other aspects, features, and examples of the present disclosure will become apparent to those of ordinary skill in the art, upon reviewing the following description of specific, exemplary aspects of the present disclosure in conjunction with the accompanying figures. While features of the present disclosure may be discussed relative to certain examples and figures below, all examples of the present disclosure can include one or more of the features discussed herein. In other words, while one or more examples may be discussed as having certain features, one or more of such features may also be used in accordance with the various examples of the disclosure discussed herein. In similar fashion, while exemplary aspects may be discussed below as device, system, or method aspects, it should be understood that such exemplary aspects can be implemented in various devices, systems, and methods.

### BRIEF DESCRIPTION OF THE DRAWINGS

FIG. 1 is a diagram illustrating an example of effective optical path length through skin according to some aspects.

FIGS. 2A and 2B are diagrams illustrating examples of apparatuses configured to control the effective optical path length through skin tissue according to some aspects.

FIGS. 3A-3D are diagrams illustrating examples of illumination optics for illuminating the skin according to some aspects.

FIGS. 4A-4D are diagrams illustrating examples of collection optics for collecting the scattered light from the skin according to some aspects.

FIG. 5 is a diagram illustrating another example of collection optics according to some aspects.

FIGS. 6A-6C are diagrams illustrating an example of illumination and collection optics according to some aspects.

FIGS. 7A and 7B are diagrams illustrating another example of illumination and collection optics according to some aspects.

FIGS. 8A and 8B are diagrams illustrating an example of illumination and collection waveguides according to some aspects.

FIGS. 9A and 9B are diagrams illustrating another example of illumination and collection waveguides according to some aspects.

FIGS. 10A and 10B are diagrams illustrating another example of illumination and

collection waveguides according to some aspects.

FIGS. 11A and 11B are diagrams illustrating an example of integration of the apparatus into a silicon chip according to some aspects.

FIGS. 12A and 12B are diagrams illustrating an example including ultrasonic transducers to reduce scattering loss according to some aspects.

FIGS. 13A-13C are diagrams illustrating an example of an apparatus including a non-dispersive infrared (ND-IR) system according to some aspects.

FIGS. 14A and 14B are diagrams illustrating an example of an apparatus using thermal effects according to some aspects.

FIG. 15 is a diagram illustrating an example of an apparatus using free space illumination optics according to some aspects.

FIGS. 16A-16C are diagrams illustrating examples of a path length control part according to some aspects.

FIGS. 17A and 17B are diagrams illustrating another example of apparatuses configured to control the effective optical path length through skin tissue on an earlobe according to some aspects.

FIGS. 18A and 18B are diagrams illustrating another example of apparatuses configured to control the effective optical path length through a finger according to some aspects.

FIG. 19 is a diagram illustrating an example of a path length control part for the tip of a finger according to some aspects.

FIG. 20 is a diagram illustrating an example of a path length control part for the bottom of a finger according to some aspects.

FIG. 21 is a diagram illustrating another example of a path length control part for the bottom of a finger according to some aspects.

FIG. 22 is a diagram illustrating an example of spectrum processing according to some aspects.

FIG. 23 is a diagram illustrating another example of a path length control part for the bottom of a finger according to some aspects.

FIGS. 24A and 24B are diagrams illustrating an example of a path length control part further configured to enable a background measurement according to some aspects.

FIGS. 25A and 25B are diagrams illustrating another example of a path length control part for the bottom of a finger according to some aspects.

FIG. 26 is a diagram illustrating an example of an apparatus including multiple detectors to measure the scattered light from the bottom and sides of the finger according to some aspects.

FIGS. 27A and 27B are diagrams illustrating another example of a path length control part according to some aspects.

FIG. 28 is a diagram illustrating an example of an apparatus configured to control the effective optical path length through skin tissue using oblique illumination according to some aspects.

FIG. 29 is a diagram illustrating examples of integration of the apparatus into a vehicle according to some aspects.

FIG. 30 is a diagram illustrating another example integration of the apparatus into a vehicle according to some aspects.

FIG. 31 is a diagram illustrating another example integration of the apparatus into a vehicle according to some aspects.

FIG. 32 is a diagram illustrating an example of an apparatus configured to control the effective optical path length through skin tissue according to some aspects.

FIG. 33 is a diagram illustrating another example of illumination and collection optics according to some aspects.

FIGS. 34A and 34B are diagrams illustrating another example of illumination and collection optics according to some aspects.

FIG. 35 is a diagram illustrating another example of an apparatus configured to control the effective optical path length through skin tissue according to some aspects.

FIGS. 36A-36C illustrate an example of filtering input light according to some aspects.

FIG. 37 is a diagram illustrating another example of illumination and collection optics according to some aspects.

FIG. 38 is a diagram illustrating another example of illumination and collection optics according to some aspects.

FIGS. 39A-39E are diagrams illustrating another example of an apparatus configured to control the effective optical path length through skin tissue according to some aspects.

FIG. 40 is a diagram illustrating an example of a skin interface of an apparatus configured to control the effective optical path length through skin tissue according to some aspects.

FIG. 41 is a diagram illustrating another example of a skin interface of an apparatus configured to control the effective optical path length through skin tissue according to some aspects.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Various aspects of the disclosure relate to mechanisms to control the effective optical path length through skin tissue for non-invasive blood biochemistry optical spectroscopy measurements. An apparatus configured for optical spectroscopy can include a path length control part configured to control the effective optical path length of diffusely scattered light non-invasively transmitted through skin tissue of a subject (e.g., human or animal). The path length control part may include, for example, a mechanical part configured to compress and hold the skin tissue to produce a controlled (e.g., optimal and/or repeatable) effective optical path length through the skin tissue. The apparatus may further include a spectral sensor and a detector (or multiple detectors). The detector (or multiple detectors) are configured to receive the diffusely scattered light and to obtain a spectrum of an analyte of the skin tissue under test based on the diffusely scattered light. In some examples, multiple detectors may be used to measure light from different skin locations or in different spectral ranges. In some examples, the spectral sensor may be an interferometer or spectrometer (e.g., a Fourier Transform infrared (FTIR) spectrometer). In some examples, the spectral sensor may include the detector. The apparatus may further include a light source configured to produce input light and direct the input light towards the path length control part or the spectral sensor. The spectral sensor is configured to either receive the input light, produce modulated light based on the input light, and direct the modulated light to the path length control part containing the skin tissue, or to receive the diffusely scattered light from the skin tissue and to obtain the spectrum using the detector. In some examples, the skin tissue includes a tip of a finger, a bottom of a fingertip, an interdigital web of a hand, an earlobe, a wrist, a nose, or a portion of a neck of the subject.

In some examples, the mechanical part includes a pressure sensor configured to measure the pressure applied by the mechanical part to the skin tissue and a pressure feedback device configured to adjust the mechanical part or notify a user to apply additional pressure based on at least one of the pressure sensor data or the spectrum (e.g., using an outlier algorithm). In some examples, the mechanical part further includes a path length measurement device configured to measure a thickness of the skin tissue corresponding to the effective optical path length. The mechanical part may further include a thickness feedback device configured to adjust the effective optical path length based on the thickness. In some examples, the apparatus may further include a processor configured to calculate a concentration of the analyte under test based on the pressure, the effective optical path length (e.g., the thickness), and the spectrum. In some examples, the mechanical part may further include a spectrum feedback device configured to receive the spectrum and adjust the effective optical path length based on the spectrum.

In some examples, the mechanical part includes an opening and walls of the opening configured to receive the skin tissue and against which pressure is applied by the subject to insert the skin tissue. For example, the mechanical part may include a pressure sensor (e.g., a spring-loaded part) configured to measure the pressure applied by the subject. The effective optical path length may then be calculated based on the pressure. In some examples, the opening includes a spring-loaded moveable diffuser in the light path of the apparatus to obtain a background spectrum prior to being locked into place by the spring-loaded part to obtain the spectrum.

In examples in which the spectral sensor is a spectrometer, the apparatus may further include a non-dispersive infrared system including a light emitting diode (LED) configured to emit light towards the skin tissue and a detector configured to receive reflected light or transmitted light from the skin tissue. In some examples, the apparatus may further include a laser source operating outside an operating range of the spectrometer and configured to illuminate the skin tissue at a wavelength corresponding to an absorption peak of the analyte. In some examples, the apparatus may further include one or more transducers configured to excite a standing acoustic wave inside the skin tissue to modify a refractive index thereof to reduce scattering loss inside the skin tissue.

In some examples, the apparatus may further include illumination optics coupled to receive incident light corresponding to the input light or the modulated light and to direct the incident light to the skin tissue in the path length control part. In some examples, the apparatus may further include collection optics configured to receive the diffusely scattered light from the skin tissue and to direct the diffusely scattered light to the spectrometer or to the detector (e.g., in examples in which the spectral sensor is an interferometer). In some examples, the illumination optics and/or collections optics may be integrated with the mechanical part. The illumination optics and collection optics may further be configured to maximize collection of light rays undergoing minimal scattering. For example, the illumination optics and collection optics may be positioned on a same axis on either side of the mechanical part. Each of the illumination optics and the collection optics may include, for example, a waveguide, a plurality of waveguides (e.g., a waveguide array), a set of one or more lenses, or a reflector.

In some examples, the illumination optics includes a plurality of waveguides. For example, the plurality of waveguides may be cleaved or non-cleaved waveguides (fibers) tilted in a horizontal plane by respective angles towards an optical axis of the collection optics. The plurality of waveguides and the collection optics may further be tilted in a vertical plan perpendicular to the optical axis by respective angles. In some examples, the plurality of waveguides are integrated into a substrate.

In some examples, the illumination optics and/or the collection optics includes a waveguide. For example, the waveguide(s) may include a dielectric or silicon slab. As another example, the waveguide(s) may include a hollow metallic slab. In this example, one or more optical windows may be included at the ends of the hollow metallic slab(s) to filter out parts of the spectrum that are not of interest for measuring the analyte to reduce heating. In addition, coupling optics may be included to provide free-space coupling of the diffusely scattered light to the spectrometer.

In some examples, the apparatus includes a silicon chip on which the illumination/collection optics and spectral sensor are integrated. For example, the spectral sensor may include a micro-electro-mechanical systems (MEMS) interferometer and the illumination and collection optics may include waveguides that are integrated into the silicon chip (e.g., fabricated into the silicon chip).

In some examples, the illumination and collection optics are fixed onto a moveable tilting component configured to tilt the illumination optics and collection optics between a first position at an angle from an optical axis of the apparatus and a second position in-plane with the optical axis of the apparatus in response to a force applied by the subject to the illumination and collection optics. In this example, the path length control part may include a latch configured to fix the illumination and collection optics in the second position to obtain the spectrum.

In some examples, the mechanical part is configured to apply mechanical pressure to a top of a finger of the subject and suction pressure to a bottom of the finger. In this example, the illumination optics may be configured to direct the input light towards the skin tissue at an oblique angle for diffused transmission of the input light through the skin tissue to produce the scattered light.

In some examples, the apparatus includes an enclosure housing the light source and including an optical window for direct illumination on the skin tissue. The apparatus may further include free space optics (e.g., within the enclosure) configured to couple the input light to the skin tissue. In some examples, the optical window may be coated with a material configured to filter a portion of the input light.

In some examples, the apparatus may be integrated into a vehicle. For example, the apparatus may be integrated into a steering wheel, an ignition press button, a console, a dashboard, or a seatbelt of the vehicle.

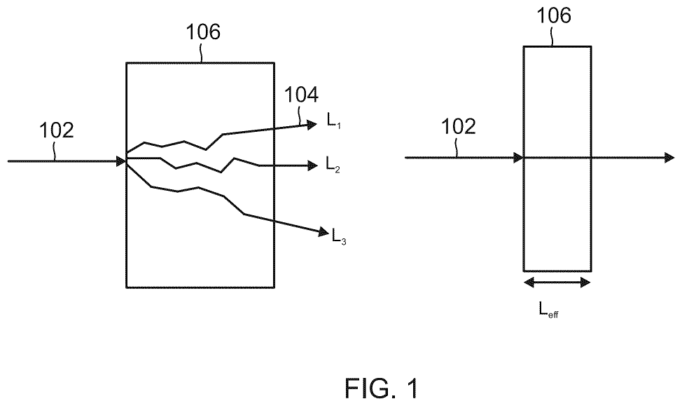

FIG. 1 is a diagram illustrating an example of effective optical path length through skin according to some aspects. In diffuse transmission, light 102 is split into many different rays 104, each taking a different path, and experiencing a different optical path length L₁, L₂, L₃ through a sample 106 (e.g., skin tissue). An effective path length L_{eff} can be calculated as a weighted average of the different path lengths L₁, L₂, L₃. In the example of skin tissue as the sample, the analyte under test is embedded in a matrix of strong optical absorption (e.g., water). Therefore, if L_{eff} is too short, the absorption of the analyte will be too small, and result in a bad limit of detection. However, if L_{eff} is too long, the absorption of the matrix will be too strong, resulting in a very low intensity of output light, and the limit of detection will be bad. An optimal L_{eff}, which is dependent upon wavelength, is possible in order to achieve the best limit of detection.

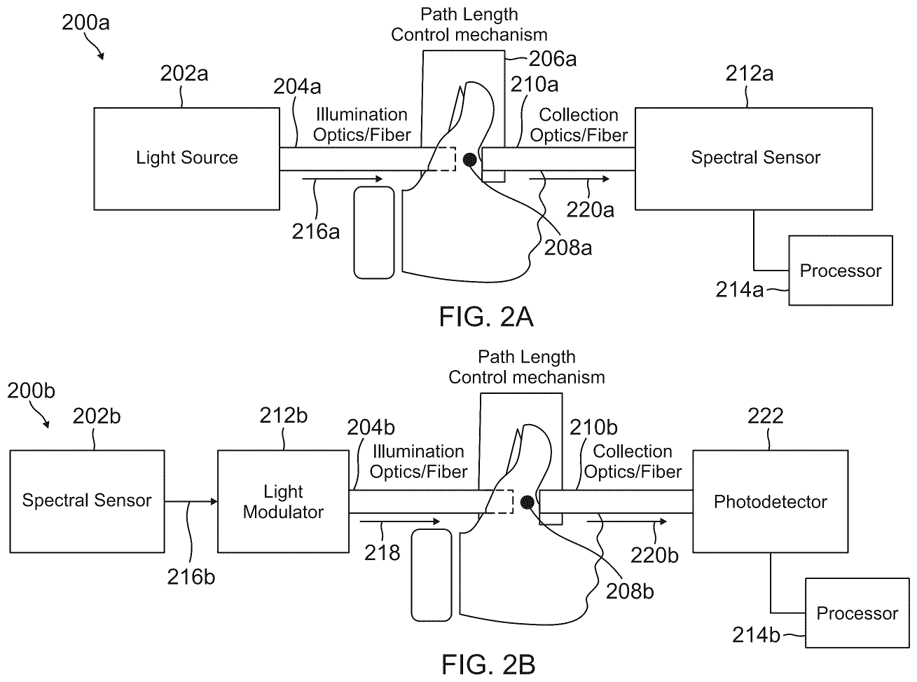

FIGS. 2A and 2B are diagrams illustrating examples of apparatuses configured to control the effective optical path length through skin tissue according to some aspects. In the examples shown in FIGS. 2A and 2B, the skin tissue corresponds to an interdigital web of a hand of a subject. The skin tissue of interdigital webs is thin enough, allowing transmission spectroscopy with adequate path length.

In the example shown in FIG. 2A, an apparatus 200a configured for non-invasive optical spectroscopy includes a light source 202a configured to generate input light 216a. The light source 202a may include, for example, a laser source, one or more wideband thermal radiation sources, or a quantum source with an array of light emitting devices that cover the wavelength range of interest. The input light 216a may be directed to illumination optics 204a configured to receive the input light 216a and to direct the input light 216a to skin tissue 208a (e.g., interdigital web) contained within a path length control part 206a. The input light 216a is transmitted through the skin tissue 208a, where the light scatters to produce diffusely scattered light 220a. The path length control part 206a is configured to control the effective optical path length of the diffusely scattered light 220a transmitted non-invasively through the skin tissue 208a to produce a target effective optical path length of the diffusely scattered light 220a through the skin tissue 208a. For example, the path length control part may be configured to compress and hold the skin tissue 208a in place during analyte measurement. The target effective optical path length may be dependent, for example, on the analyte (e.g., blood biochemical or biomarker) of the skin tissue 208a under test. For example, the target effective optical path length may be dependent upon the wavelength of interest for the biomarker. In some examples, the target effective optical path length has little dependence on the analyte scattering, absorption, or wavelength of the input light. In some examples, the target effective optical path length may be an optimal effective optical path length for diffuse transmission.

The diffusely scattered light 220a output from the skin tissue 208a is coupled to collection optics 210a configured to receive the diffusely scattered light 220a and direct the diffusely scattered light 220a to a spectral sensor 212a. The illumination optics 204a and collection optics 210a may further be configured to maximize collection of light rays undergoing minimal scattering. For example, the illumination optics and collection optics may be positioned on a same axis on either side of the path length control part 206a. The spectral sensor 212a shown in FIG. 2A may be, for example, a spectrometer including a detector to obtain a spectrum of the analyte under test. The spectrometer 212a may include, for example, a Fourier Transform infrared (FTIR) spectrometer that exploits light interference and Fourier transform to produce a spectrum of the analyte under test. For example, the spectrometer 212a may include a Michelson FTIR interferometer in which the spectrum may be retrieved, for example, using a Fourier transform carried out by a processor 214a. The spectrometer 212a is not limited to a Michelson FTIR interferometer, and may include any spectrometer type, such as a Fabry-Perot spectrometer, a diffraction grating spectrometer, or other suitable type of spectrometer.

In the example shown in FIG. 2B, an apparatus 200b configured for non-invasive optical spectroscopy includes a light source 202b configured to generate input light 216b. The input light 216b may be, for example, multi-wavelength light. The light source 202b may include, for example, a laser source, one or more wideband thermal radiation sources, or a quantum source with an array of light emitting devices that cover the wavelength range of interest. The input light 216b may be directed to a spectral sensor 212b configured to receive the input light 216b and to produce modulated light 218 based on the input light 216b. For example, the spectral sensor 212b may be an interferometer (e.g., a Michelson interferometer) or other suitable type of spectral sensor, such as a diffraction element, a Fabry-Perot cavity, a spatial spectral sensor, or a birefringent device.

The modulated light 218 may be directed to illumination optics 204a configured to receive the modulated light 218 and to direct the modulated light 218 to skin tissue 208b (e.g., interdigital web) contained within a path length control part 206b. The modulated light 218 is transmitted through the skin tissue 208b, where the light scatters to produce diffusely scattered light 220b. The path length control part 206a is configured to control the effective optical path length of the diffusely scattered light 220b transmitted through the skin tissue 208b to produce a target effective optical path length of the diffusely scattered light 220b through the skin tissue 208b, as described above.

The diffusely scattered light 220b output from the skin tissue 208b is coupled to collection optics 210b configured to receive the diffusely scattered light 220b and direct the diffusely scattered light 220b to a detector 222 (e.g., a photodetector) to obtain a spectrum of the analyte under test. In some examples, the detector 222 may include a single detector, a detector array, or a multi-pixel detector. In examples in which the detector 222 includes multiple detectors, each detector may be configured to measure light from different skin locations or in different spectral ranges. In examples in which the spectral sensor 212b is a FT-IR spectrometer or Fabry-Perot spectrometer, the modulated light 218 may correspond to interference beams produced over time with an OPD between beams. The output of the detector 222 may then correspond to an interferogram, which may be input to a processor 214b to retrieve the spectrum. In examples in which the spectral sensor 212b is a diffraction grating, the modulated light 218 may correspond to diffracted light across a plurality of wavelengths. The output of the detector 222 may then correspond to an image representing the light intensity at each wavelength point on the detector, which may be input to the processor 214b to retrieve the spectrum. As in the example shown in FIG. 2A, the illumination optics 204b and collection optics 210b may further be configured to maximize collection of light rays undergoing minimal scattering. For example, the illumination optics and collection optics may be positioned on a same axis on either side of the path length control part 206b.

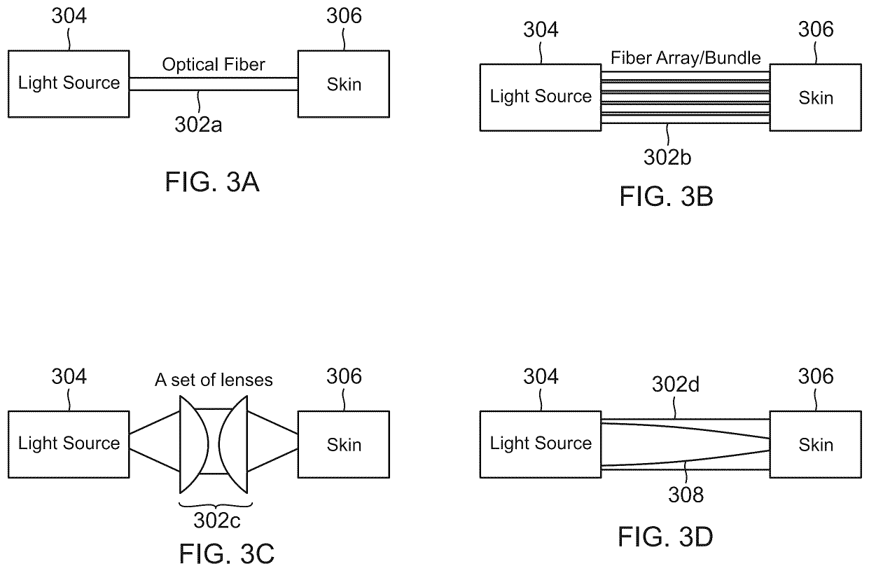

FIGS. 3A-3D are diagrams illustrating examples of illumination optics for illuminating the skin according to some aspects. In the example shown in FIG. 3A, the illumination optics can include an optical fiber 302a optically coupled to direct input light (or modulated light) from a light source 304 (or a spectral sensor) to skin tissue 306. In the example shown in FIG. 3B, the illumination optics can include a plurality of waveguides (e.g., a waveguide array) 302b optically coupled to direct input light (or modulated light) from the light source 304 (or spectral sensor) to skin tissue 306. In the example shown in FIG. 3C, the illumination optics can include a set of one or more lenses 302c (two of which are shown) optically coupled to direct input light (or modulated light) from the light source 304 (or spectral sensor) to skin tissue 306. In the example shown in FIG. 3D, the illumination optics can include a reflector 302d optically coupled to direct input light (or modulated light) from the light source 304 (or spectral sensor) to skin tissue 306. In some examples, the reflector 302d can include a metallized molded part 308 having a shape forming a compound parabolic concentrator (CPC) or a compound elliptic concentrator (CEC).

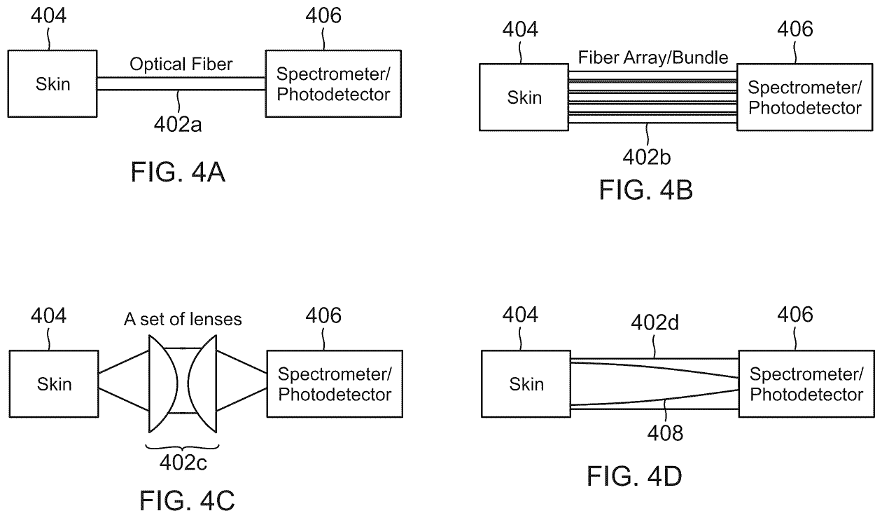

FIGS. 4A-4D are diagrams illustrating examples of collection optics for collecting the diffusely scattered light from the skin according to some aspects. In the example shown in FIG. 4A, the collection optics can include an optical fiber 402a optically coupled to direct diffusely scattered light from skin tissue 404 to a spectrometer/photodetector 406. In the example shown in FIG. 4B, the collection optics can include a plurality of waveguides (e.g., a waveguide array) 402b optically coupled to direct diffusely scattered light from skin tissue 404 to a spectrometer/photodetector 406. In the example shown in FIG. 4C, the collection optics can include a set of one or more lenses 402c (two of which are shown) optically coupled to direct diffusely scattered light from skin tissue 404 to a spectrometer/photodetector 406. In the example shown in FIG. 4D, the collection optics can include a reflector 402d optically coupled to direct diffusely scattered light from skin tissue 404 to a spectrometer/photodetector 406. In some examples, the reflector 402d can include a metallized molded part 408 having a shape forming a CPC or a CEC.

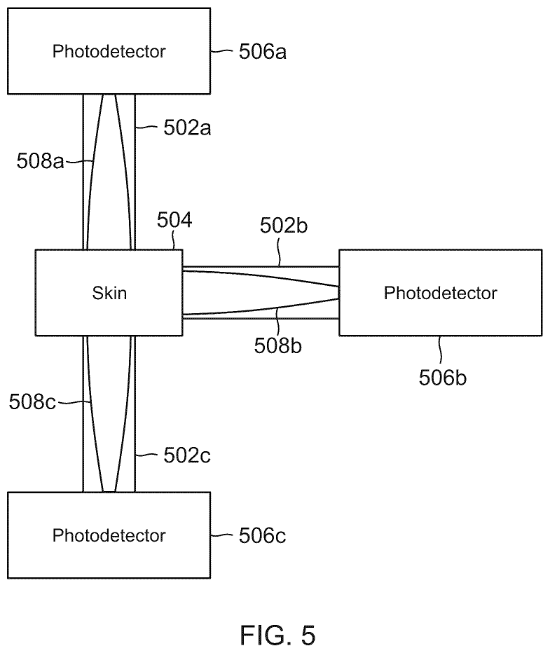

FIG. 5 is a diagram illustrating another example of collection optics according to some aspects. In the example shown in FIG. 5, the collection optics can include an array of reflectors or concentrators 502a, 502b, and 502c optically coupled to collect diffusely scattered light from different parts of the skin tissue 504 and to direct the diffusely scattered light to respective detectors (photodetectors) 506a, 506b, and 506c. Thus, in this example, the collection optics includes a set of two or more reflectors (three of which are shown) 502a, 502b, and 502c, each configured to direct diffusely scattered light to a corresponding respective detector (again, three of which are shown) 506a, 506b, and 506c. In some examples, the reflectors 502a, 502b, and 502c can each include a respective metallized molded part 508a, 508b, and 508c having a shape forming a CPC or a CEC. In some examples, the detectors 506a, 506b, and 506c may further be configured to each have a different respective spectral range.

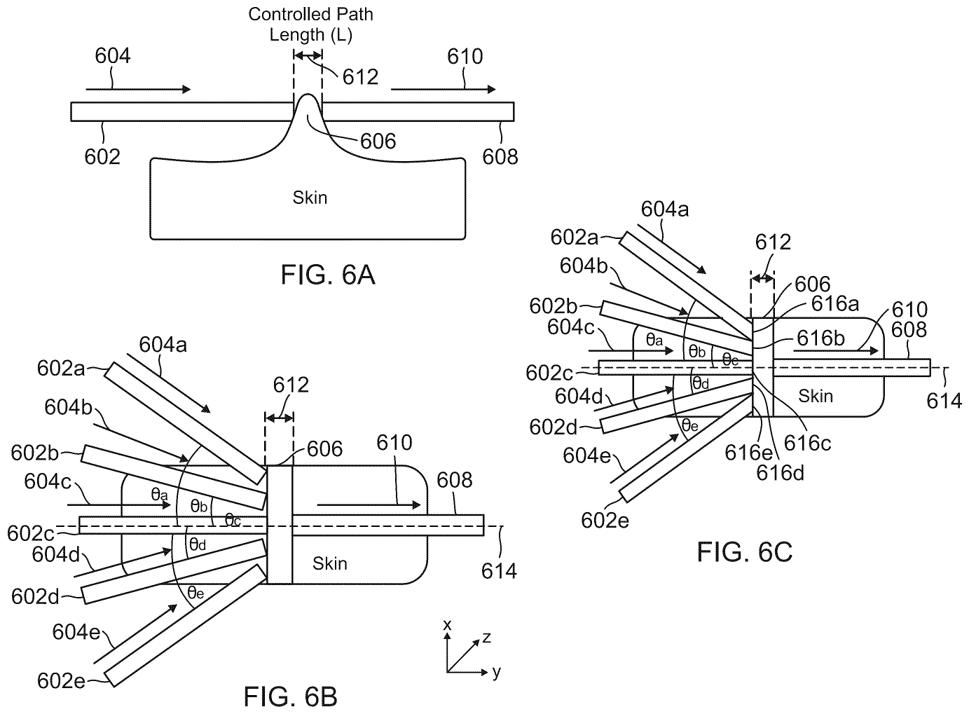

FIGS. 6A-6C are diagrams illustrating an example of illumination and collection optics according to some aspects. FIG. 6A is a side view of the illumination and collection optics, while FIGS. 6B and 6C are top views of different configurations of the illumination and collection optics. As shown in FIG. 6A, illumination optics 602 are optically coupled to direct input light (or modulated light) 604 to skin tissue 606. The light (input or modulated) 604 is transmitted through the skin tissue 606 to produce diffusely scattered light 610 that is collected by collection optics 608. The input/modulated light 604 is transmitted through the skin tissue 606 with a target effective optical path length (L) 612 produced by a path length control part (not shown). For example, the skin tissue 606 may be compressed and held at a thickness selected to produce a target effective path length.

As shown in FIGS. 6A and 6B, the illumination optics 602 can include a plurality of waveguides (optical fibers) 602a-602e, each configured to direct a respective portion of the light (input or modulated) 604a-604e towards the skin tissue 606. Each of the waveguides 602a-602e is tilted in a horizontal plane (x-y plane) by respective angles θₐ-θₑ towards an optical axis 614 of the collection optics 608. The tilting of the waveguides 602a-602e may enable more light to be coupled into the collection optics 608. In the example shown in FIG. 6C, the waveguides 602a-602e may be angle-cleaved optical fibers to maintain the fiber tips 616a-616e in contact with the skin tissue 606 while tilted. As used herein, the term angle-cleaved may refer to any angle between 0 and 180 degrees.

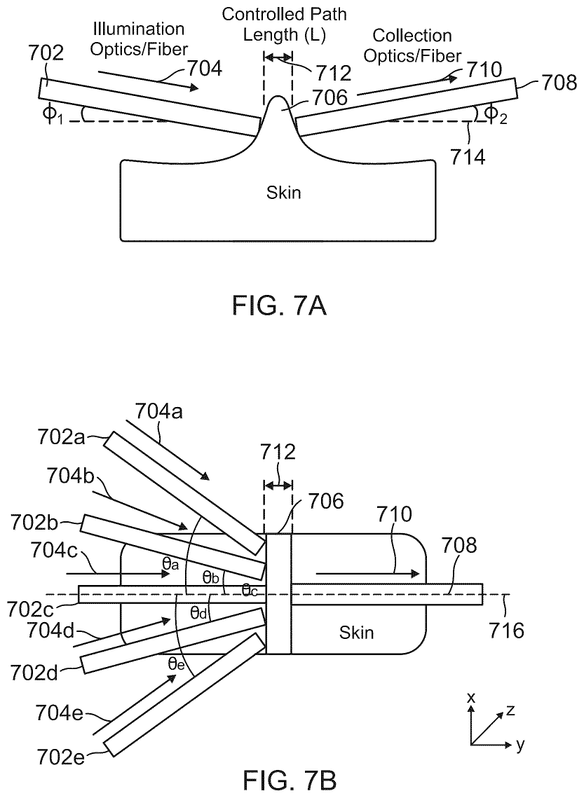

FIGS. 7A and 7B are diagrams illustrating another example of illumination and collection optics according to some aspects. FIG. 7A is a side view of the illumination and collection optics, while FIG. 7B is a top view of the illumination and collection optics. As shown in FIG. 7A, illumination optics 702 are optically coupled to direct input light (or modulated light) 704 to skin tissue 706. The light (input or modulated) 704 is transmitted through the skin tissue 706 to produce diffusely scattered light 710 that is collected by collection optics 708. The input/modulated light 704 is transmitted through the skin tissue 706 with a target effective optical path length (L) 712 produced by a path length control part (not shown). For example, the skin tissue 706 may be compressed and held at a thickness selected to produce a target effective path length.

In addition, as illustrated in FIG. 7A, the illumination optics 702 and the collection optics 708 may each be tilted in a vertical plane (z axis) that is perpendicular to an optical axis 714 of the diffusely scattered light transmitted through the skin tissue 706 by respective angles (ϕ₁ and ϕ₂). As shown in FIG. 7B, the illumination optics 702 can further include a plurality of waveguides (optical fibers) 702a-702e, each configured to direct a respective portion of the light (input or modulated) 704a-704e towards the skin tissue 706. Each of the waveguides 702a-702e can also be tilted in a horizontal plane (x-y plane) by respective angles θₐ-θₑ towards an optical axis 716 of the collection optics 708. In some examples, the waveguides 702a-702e may be angle-cleaved optical fibers, for example, as shown in FIG. 6C.

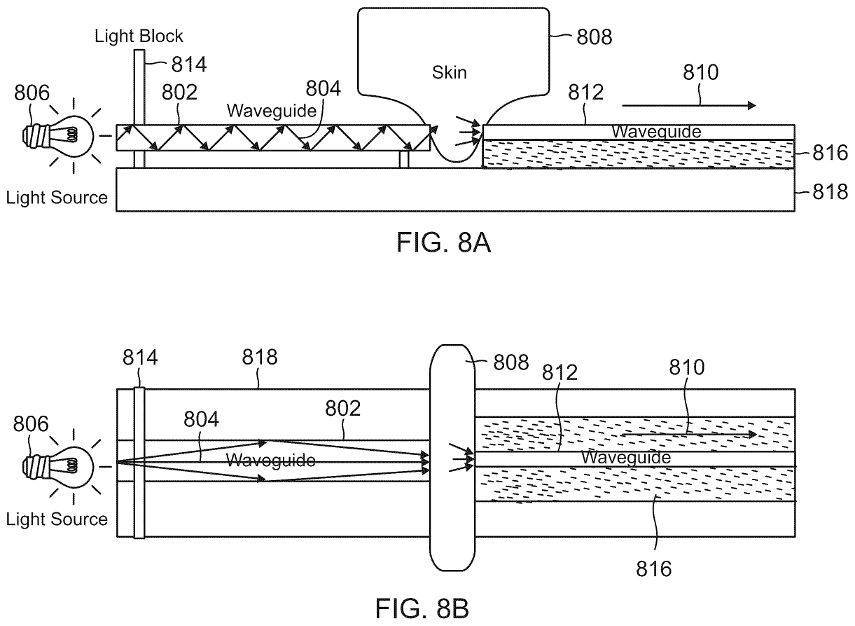

FIGS. 8A and 8B are diagrams illustrating an example of illumination and collection waveguides according to some aspects. FIG. 8A is a side view of the illumination and collection waveguides, while FIG. 8B is a top view of the illumination and collection waveguides. In the example shown in FIGS. 8A and 8B, an illumination waveguide 802 is optically coupled to direct input light 804 from a light source 806 (or modulated light from a spectral sensor) to skin tissue 808. The light (input or modulated) 804 is transmitted through the skin tissue 808 to produce diffusely scattered light 810 that is collected by a collection waveguide 812.

In some examples, the illumination waveguide 802 may be a slab waveguide, such as dielectric, glass, sapphire, or silicon slab waveguides. For example, the illumination waveguide 802 may be in the form of a dielectric slab which guides the light 804 to the surface of the skin tissue 808. As another example, the illumination waveguide 802 may be in the form of a silicon slab to act as an optical filter to filter shorter wavelengths to reduce the skin tissue heating. Dielectric slabs may have a lower cost and easier assembly as compared to, for example, an optical fiber. In addition, a slab waveguide may have a higher throughput, as it may have a larger area and/or numerical aperture as compared to optical fiber. A light block component 814 may further be integrated onto the surface of the illumination waveguide 802 to prevent stray light from reflecting back onto light source 806.

In some examples, the collection waveguide 812 may be an optical fiber mounted on or otherwise positioned on a mechanical support 816. In other examples, the collection waveguide 812 may further be a slab waveguide, such as a dielectric, glass, sapphire, or silicon slab waveguide. In some examples, the illumination waveguide 802 and collection waveguide 812 may be integrated on a substrate 818, such as a silicon, glass, or other suitable substrate.

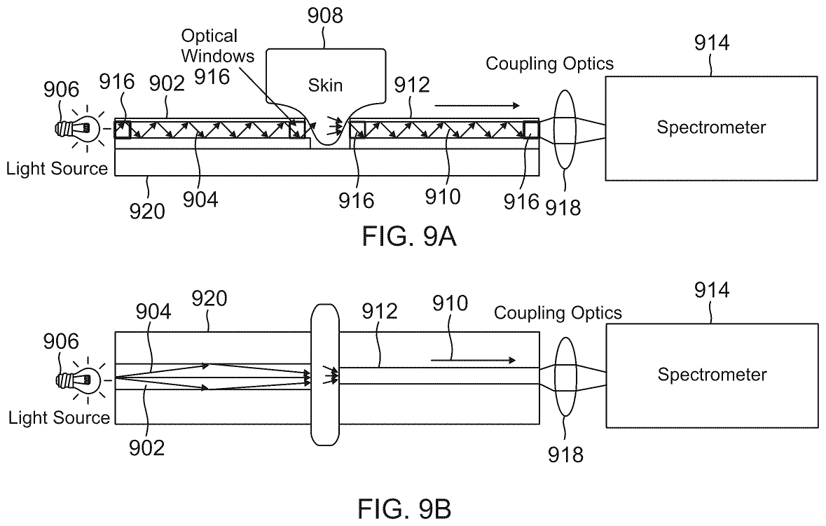

FIGS. 9A and 9B are diagrams illustrating another example of illumination and collection waveguides according to some aspects. FIG. 9A is a side view of the illumination and collection waveguides, while FIG. 9B is a top view of the illumination and collection waveguides. In the example shown in FIGS. 9A and 9B, an illumination waveguide 902 is optically coupled to direct input light 904 from a light source 906 (or modulated light from a spectral sensor) to skin tissue 908. The light (input or modulated) 904 is transmitted through the skin tissue 908 to produce diffusely scattered light 910 that is collected by a collection waveguide 912 and directed towards a spectrometer/photodetector 914. In some examples, coupling optics 918 (e.g., free-space coupling optics) may be used to couple an output of the collection waveguide 912 (e.g., the diffusely scattered light 910) to the spectrometer/photodetector 914.

In some examples, the illumination waveguide 902 and/or the collection waveguide 912 may be a hollow metallic waveguide. The hollow metallic waveguides 902 and 912 may further be integrated on a substrate 920. In addition, one or more optical windows 916 may be coupled to the hollow metallic slab 902 and/or 912 (e.g., input or output of the hollow metallic slab 902 and/or 912) to filter the light 904 and/or 910. For example, the optical window(s) 916 may be configured to filter out part(s) of the spectrum that are not of interest for measuring the analyte under test to reduce heating. In some examples, the optical window(s) 916 may include a coating designed to filter the undesired portion(s) of the spectrum. In some examples, the optical window(s) 916 may be fabricated of a material having an absorption designed to filter the undesired portion(s) of the spectrum.

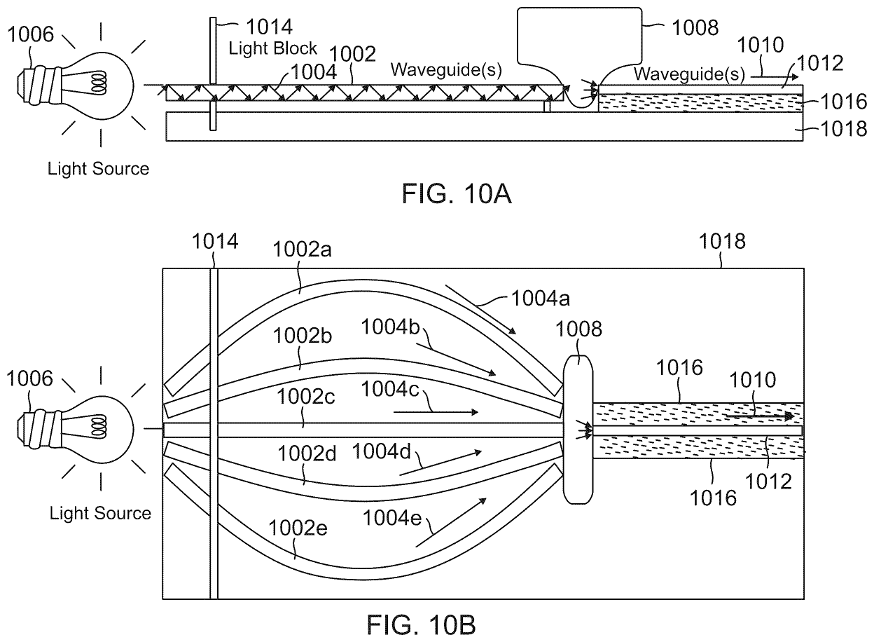

FIGS. 10A and 10B are diagrams illustrating another example of illumination and collection waveguides according to some aspects. FIG. 10A is a side view of the illumination and collection waveguides, while FIG. 10B is a top view of the illumination and collection waveguides. In the example shown in FIGS. 10A and 10B, an illumination waveguide 1002 is optically coupled to direct input light 1004 from a light source 1006 (or modulated light from a spectral sensor) to skin tissue 1008. The light (input or modulated) 1004 is transmitted through the skin tissue 1008 to produce diffusely scattered light 1010 that is collected by a collection waveguide 1012. A light block component 1014 may further be integrated onto the surface of the illumination waveguide 1002 to prevent stray light from reflecting back onto light source 1006.

As shown in FIG. 10B, the illumination waveguide 1002 can further include a plurality of waveguides (optical fibers, hollow metallic waveguides, etc.) 1002a-1002e, each configured to direct a respective portion of the light (input or modulated) 1004a-1004e towards the skin tissue 1008, similar to that shown in FIG. 6B/6C or 7B. Thus, the plurality of waveguides 1002a-1002e may be configured to illuminate the skin tissue 1008 at different angles on the skin. In some examples, the collection waveguide 1012 may be mounted on or otherwise positioned on a mechanical support 1016. In some examples, the illumination waveguide 1002 and collection waveguide 1012 may be integrated on a substrate 1018.

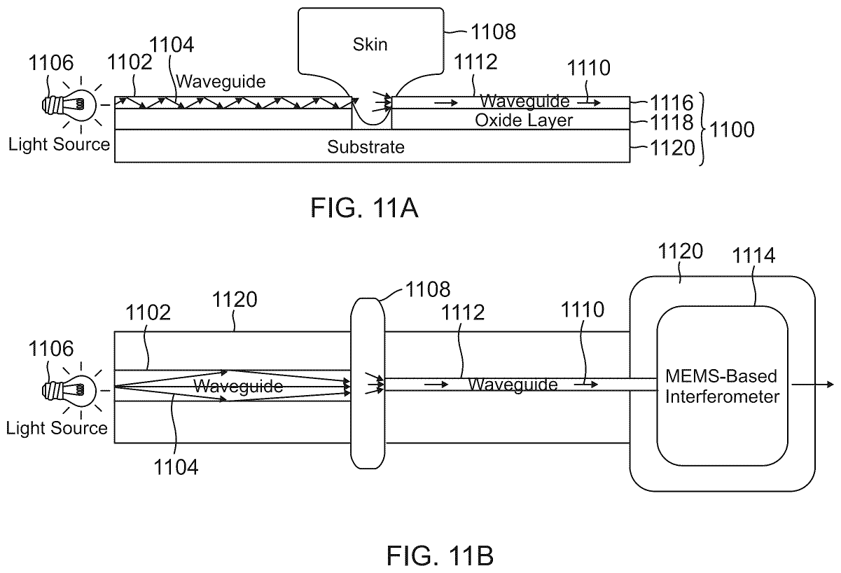

FIGS. 11A and 11B are diagrams illustrating an example of integration of the apparatus into a silicon chip 1100 according to some aspects. FIG. 11A is a side view of the illumination and collection waveguides, while FIG. 11B is a top view of the illumination and collection waveguides. In the example shown in FIGS. 11A and 11B, an illumination waveguide 1102 is optically coupled to direct input light 1104 from a light source 1106 to skin tissue 1108. The input light 1104 is transmitted through the skin tissue 1108 to produce diffusely scattered light 1110 that is collected by a collection waveguide 1112 and directed to a spectrometer 1114. In the example shown in FIG. 11B, the spectrometer 1114 may be, for example, a MEMS-based interferometer that is integrated in (e.g., fabricated within) the silicon chip 1100. The silicon chip 1100 may be, for example, a silicon-on-insulator (SOI) chip including a device layer 1116, a substrate 1120 (e.g., silicon substrate forming a handle layer), and an oxide layer 1118 (e.g., silicon dioxide) sandwiches between the device layer 1116 and the handle layer 1120. The illumination waveguide 1102, collection waveguide 1112, and MEMS-based interferometer 1114 may each be integrated and fabricated on the silicon chip 1100 (e.g., fabricated within the device layer 1116 of the silicon chip 1100).

In some examples, the MEMS-based interferometer 1114 may further be attached to a printed circuit board (PCB) that may include, for example, one or more processors, memory devices, buses, and/or other components. As used herein, the term MEMS refers to an actuator, a sensor, or the integration of sensors, actuators and electronics on a common silicon substrate through microfabrication technology to build a functional system. Microelectronics are typically fabricated using an integrated circuit (IC) process, while the micromechanical components are fabricated using compatible micromachining processes that selectively etch away parts of the silicon wafer or add new structural layers to form the mechanical and electromechanical components. One example of a MEMS element is a micro-optical component having a dielectric or metallized surface working in a reflection or refraction mode. Other examples of MEMS elements include actuators, detector grooves and fiber grooves (e.g., for the illumination and/or collection waveguides 1102 and 1112).

In some examples, the MEMS interferometer 1114 may be fabricated using a Deep Reactive Ion Etching (DRIE) process on the silicon chip (e.g., as part of an SOI wafer) in order to produce the micro-optical components and other MEMS elements that are able to process free-space optical beams propagating parallel to the SOI substrate. For example, the electro-mechanical designs may be printed on masks and the masks may be used to pattern the design over the silicon or SOI wafer by photolithography. The patterns may then be etched (e.g., by DRIE) using batch processes, and the resulting chips (e.g., MEMS silicon chips 1100) may be diced and packaged (e.g., attached to the PCB).

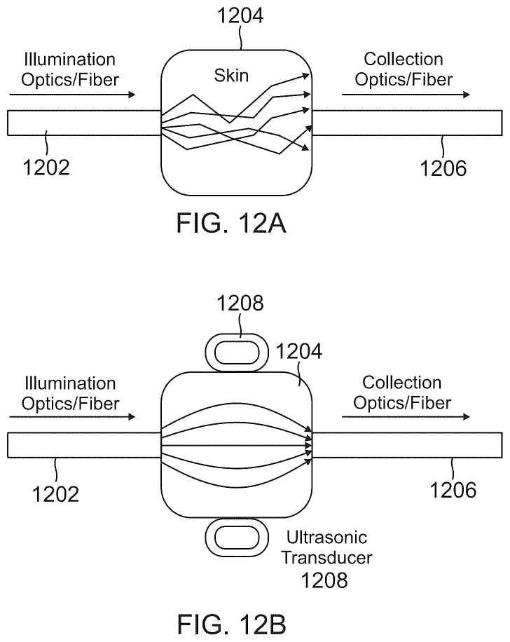

FIGS. 12A and 12B are diagrams illustrating an example including ultrasonic transducers to reduce scattering loss according to some aspects. As shown in FIG. 12A, due to scattering effects inside the skin tissue 1204 under test, the light scatters randomly in all directions, and only a fraction of the input light from the illumination optics 1202 can be coupled to the throughput-limited collection optics 1206. This may lead to very large losses in the light signal. As shown in FIG. 12B, this scattering loss may be reduced using ultrasonic transducers 1208 that excite a standing acoustic wave inside the skin tissue 1204 to modify a refractive index of the skin tissue 1204 that enables the light to act as a lens inside the skin tissue 1204 to reduce the scattering loss of the input light.

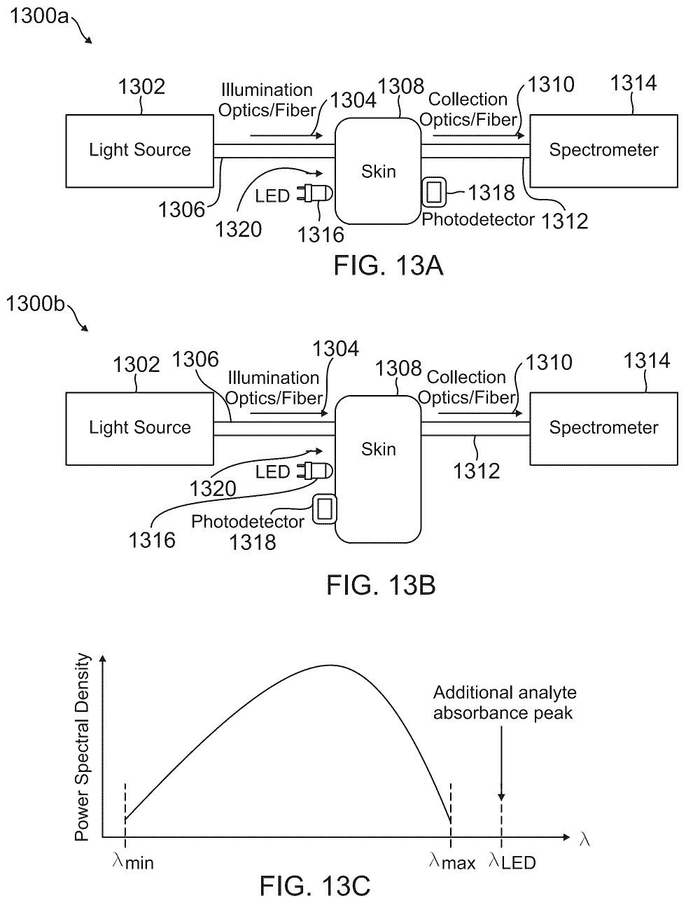

FIGS. 13A-13C are diagrams illustrating an example of an apparatus including a non-dispersive infrared (ND-IR) system according to some aspects. The apparatus 1300a/1300b includes a light source 1302 configured to emit input light 1304 and illumination optics 1306 configured to direct the input light 1304 to skin tissue 1308 under test for transmission through the skin tissue 1308 to produce diffusely scattered light 1310. The apparatus 1300a/1300b further includes collection optics 1312 configured to receive the diffusely scattered light 1310 from the skin tissue 1308 and to direct the diffusely scattered light 1310 to a spectral sensor 1314 (e.g., a spectrometer including a detector).

The apparatus 1300a/1300b further includes one or more ND-IR systems (one of which is shown), each including at least one narrowband light source (e.g., light emitting diode (LED)) 1316 configured to emit additional light 1320 towards the skin tissue 1308 and at least one detector 1318 (e.g., photodetector) configured to receive light from the skin tissue 1308. In the example shown in FIG. 13A, the photodetector 1318 may be configured to receive light transmitted through the skin tissue 1308 in a transmission mode, whereas in FIG. 13B, the photodetector 1318 may be configured to receive light reflected from the skin tissue 1308 in a reflection mode. The ND-IR system(s) can be used in addition to the spectrometer 1314 to aid in detecting spectral features outside the operating range of the spectrometer 1314. As shown in FIG. 13C, the LED(s) 1316 and photodetector(s) 1318 may be configured to detect the skin optical absorbance signal at wavelength(s) ALED outside the operating spectral range (λₘᵢₙ to λₘₐₓ) of the spectrometer 1314. For example, a spectrometer 1314 with a maximum operating wavelength of 1900 nm may be used alongside an LED 1316 and photodetector 1318 system operating at 2300 nm to detect the ethanol absorption peak at 2300 nm.

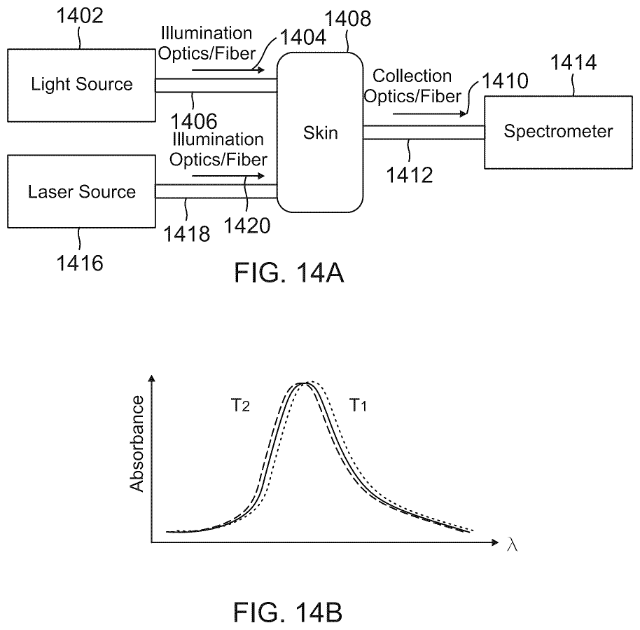

FIGS. 14A and 14B are diagrams illustrating an example of an apparatus using thermal effects according to some aspects. The apparatus 1400 includes a light source 1402 configured to emit input light 1404 and illumination optics 1406 configured to direct the input light 1404 to skin tissue 1408 under test for transmission through the skin tissue 1408 to produce diffusely scattered light 1410. The apparatus 1400 further includes collection optics 1412 configured to receive the diffusely scattered light 1410 from the skin tissue 1408 and to direct the diffusely scattered light 1410 to a spectral sensor 1414 (e.g., a spectrometer including a detector).

The apparatus 1400 further includes a laser source 1416 (e.g., a mid-infrared laser source) configured to generate additional light 1420 and illuminate the skin tissue 1408 at a wavelength outside an operating range of the spectrometer 1414 and corresponding to an absorption peak of an analyte under test. Additional illumination optics 1418 may be configured to direct the additional light 1420 towards the skin tissue 1408. As shown in FIG. 14B, the laser source 1416 induces a heating effect on the skin tissue 1408 that is dependent upon the concentration of the analyte in the skin tissue 1408. The heat then causes a shift in the water absorbance spectrum (T₁ to T₂) obtained by the spectrometer 1414. Thus, the spectrometer 1414 can be used to measure the water absorbance spectrum and calculate the analyte concentration using, for example, a processor (e.g., an algorithm executed by the processor).

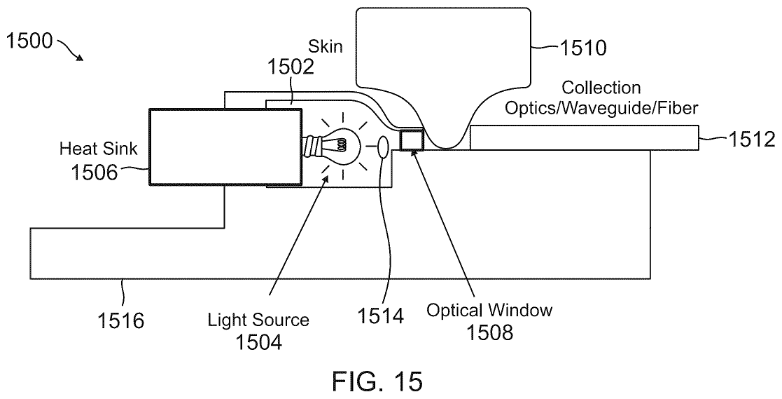

FIG. 15 is a diagram illustrating an example of an apparatus 1500 using free space illumination optics according to some aspects. In the example shown in FIG. 15, the apparatus 1500 includes an enclosure 1502 housing a light source 1504. The enclosure 1502 may further include an optical window 1508 for direct illumination on skin tissue 1510. In some examples, the apparatus 1500 may further include free space optics 1514 (e.g., within the enclosure 1502) configured to couple (or direct) the input light from the light source 1504 through the optical window 1508 to the skin tissue. Diffusely scattered light produced by the skin tissue 1510 may be coupled to collection optics 1512. In some examples, the enclosure 1502 and collection optics 1512 may be integrated on a substrate 1516. A heat sink 1506 may further be coupled to the light source 1504 to minimize heating of the skin tissue 1510. In some examples, the optical window 1508 may be configured to filter out part(s) of the spectrum that are not of interest for measuring the analyte under test to further reduce heating. For example, the optical window 1508 may be coated with a material configured to filter out portion(s) of the spectrum or may be fabricated by a window material designed to filter the undesired portion(s) of the spectrum.

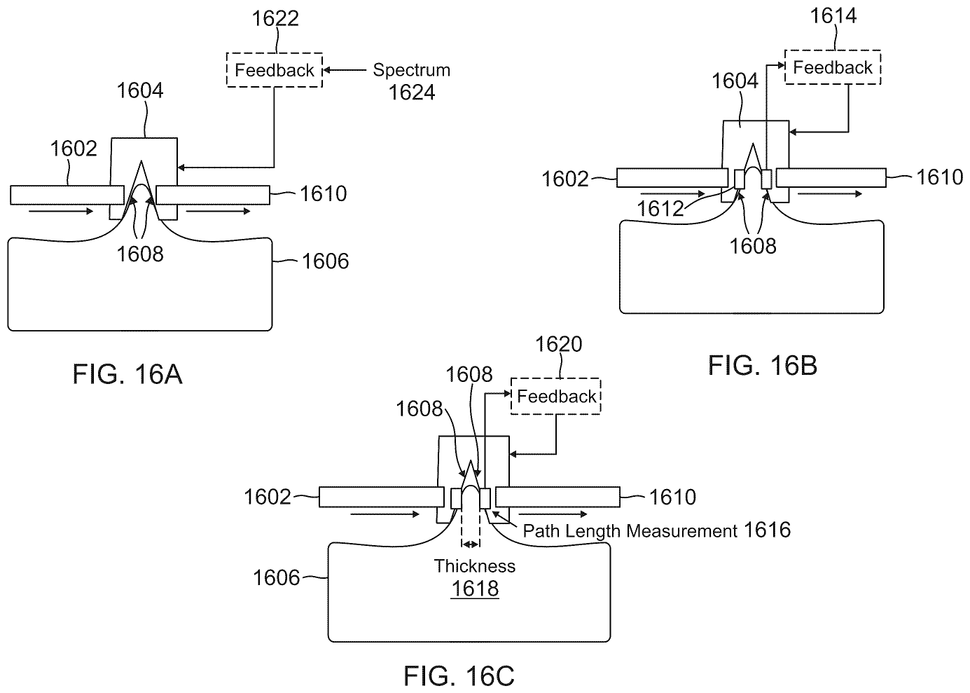

FIGS. 16A-16C are diagrams illustrating examples of a path length control part according to some aspects. In the example shown in FIGS. 16A-16C, a path length control part 1604 includes a mechanical part 1608 configured to compress and hold skin tissue 1606 under test to produce a target effective optical path length through the skin tissue 1606. The mechanical part 1608 may further be configured to produce a fixed effective optical path length repeatable across respective (e.g., successive) measurements of the skin tissue 1606. For example, the mechanical part may further include a spectrum feedback device 1622 configured to receive the spectrum 1624 and adjust the effective optical path length based on the spectrum 1624. The mechanical part 1608 may include a portion of illumination optics 1602 and/or collection optics 1610 (e.g., respective ends of the illumination and/or collection optics 1602 and 1610 on either side of the skin tissue 1606). Thus, the illumination optics 1602 and/or collection optics 1610 may be integrated into the mechanical part 1608 (e.g., mechanical skin tissue holder).

In the example shown in FIG. 16B, the mechanical part 1608 may further include a pressure sensor 1612 configured to measure a pressure applied by the mechanical part 1608 to the skin tissue 1606 to produce pressure sensor data. A feedback device 1614 may further be coupled with the mechanical part 1608 to adjust the mechanical part 1608 or notify a user to apply additional pressure based on the pressure sensor data or the spectrum (e.g., using an outlier algorithm). By adjusting the mechanical part 1608 or notifying the user to apply additional pressure, the feedback device 1614 can be configured to control and adjust the effective optimal path length to produce the target effective optical path length.

In the example shown in FIG. 16C, the mechanical part 1608 may further include a path length measurement device 1616 configured to measure a thickness 1618 of the skin tissue corresponding to the effective optical path length. A feedback device 1620 may further be coupled with the mechanical part 1608 to adjust the mechanical part 1608 based on the thickness 1618. By adjusting the mechanical part 1608 based on the thickness, the feedback device 1620 can be configured to control and adjust the effective optimal path length to produce the target effective optical path length.

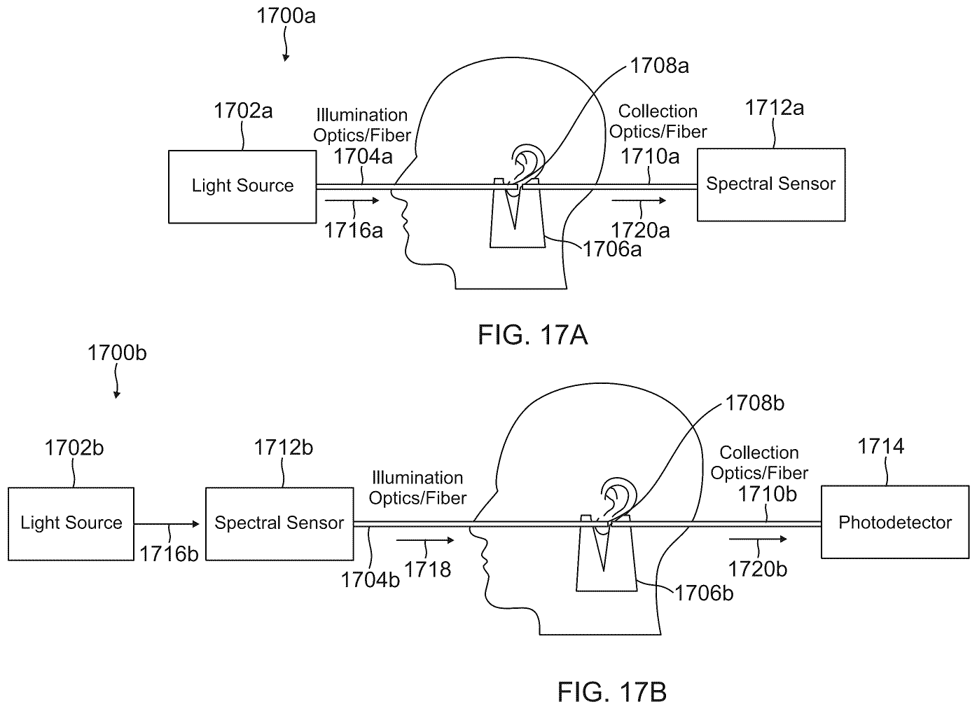

FIGS. 17A and 17B are diagrams illustrating another example of apparatuses configured to control the effective optical path length through skin tissue on an earlobe according to some aspects. Earlobes also have the advantage of being thin enough, allowing transmission spectroscopy with optimal path length.

In the example shown in FIG. 17A, an apparatus 1700a configured for non-invasive optical spectroscopy includes a light source 1702a configured to generate input light 1716a. The light source 1702a may include, for example, a laser source, one or more wideband thermal radiation sources, or a quantum source with an array of light emitting devices that cover the wavelength range of interest. The input light 1716a may be directed to illumination optics 1704a configured to receive the input light 1716a and to direct the input light 1716a to skin tissue 1708a (e.g., earlobe) contained within a path length control part 1706a. The input light 1716a is transmitted through the skin tissue 1708a, where the light scatters to produce diffusely scattered light 1720a. The path length control part 1706a is configured to control the effective optical path length of the diffusely scattered light 1720a transmitted through the skin tissue 1708a to produce a target effective optical path length of the diffusely scattered light 1720a through the skin tissue 1708a. For example, the path length control part may be configured to compress and hold the skin tissue 1708a in place during analyte measurement. The target effective optical path length may be dependent, for example, on the analyte (e.g., blood biochemical or biomarker) of the skin tissue 1708a under test. For example, the target effective path length may be dependent upon the wavelength of interest for the biomarker.

The diffusely scattered light 1720a output from the skin tissue 1708a is coupled to collection optics 1710a configured to receive the diffusely scattered light 1720a and direct the diffusely scattered light 1720a to a spectral sensor 1712a. The spectral sensor 1712a shown in FIG. 17A may be, for example, a spectrometer including a detector to obtain a spectrum of the analyte under test. The spectrometer 1712a may include, for example, a Fourier Transform infrared (FTIR) spectrometer that exploits light interference and Fourier transform to produce a spectrum of the analyte under test. For example, the spectrometer 1712a may include a Michelson FTIR interferometer in which the spectrum may be retrieved, for example, using a Fourier transform carried out by a processor (not shown). The spectrometer 1712a is not limited to a Michelson FTIR interferometer, and may include any spectrometer type, such as a Fabry-Perot spectrometer, a diffraction grating spectrometer, or other suitable type of spectrometer.

In the example shown in FIG. 17B, an apparatus 1700b configured for non-invasive optical spectroscopy includes a light source 1702b configured to generate input light 1716b. The light source 1702b may include, for example, a laser source, one or more wideband thermal radiation sources, or a quantum source with an array of light emitting devices that cover the wavelength range of interest. The input light 1716b may be directed to a spectral sensor 1712b configured to receive the input light 1716b and to produce modulated light 1718 based on the input light 1716b. For example, the spectral sensor 1712b may be an interferometer (e.g., a Michelson interferometer) or other suitable type of spectral sensor, such as a diffraction element, a Fabry-Perot cavity, a spatial spectral sensor, or a birefringent device.

The modulated light 1718 may be directed to illumination optics 1704a configured to receive the modulated light 1718 and to direct the modulated light 1718 to skin tissue 1708b (e.g., earlobe) contained within a path length control part 1706b. The modulated light 1718 is transmitted through the skin tissue 1708b, where the light scatters to produce diffusely scattered light 1720b. The path length control part 1706a is configured to control the effective optical path length of the diffusely scattered light 1720b transmitted through the skin tissue 1708b to produce a target effective optical path length of the diffusely scattered light 1720b through the skin tissue 1708b, as described above.

The diffusely scattered light 1720b output from the skin tissue 1708b is coupled to collection optics 1710b configured to receive the diffusely scattered light 1720b and direct the diffusely scattered light 1720b to a detector 1714 (e.g., a photodetector) to obtain a spectrum of the analyte under test. In some examples, the detector 1714 may include a single detector, a detector array, or a multi-pixel detector. In examples in which the spectral sensor 1712b is a FT-IR spectrometer or Fabry-Perot spectrometer, the modulated light 1718 may correspond to interference beams produced over time with an OPD between beams. The output of the detector 1714 may then correspond to an interferogram, which may be input to a processor (not shown) to retrieve the spectrum. In examples in which the spectral sensor 1712b is a diffraction grating, the modulated light 1718 may correspond to diffracted light across a plurality of wavelengths. The output of the detector 1714 may then correspond to an image representing the light intensity at each wavelength point on the detector, which may be input to the processor (not shown) to retrieve the spectrum.

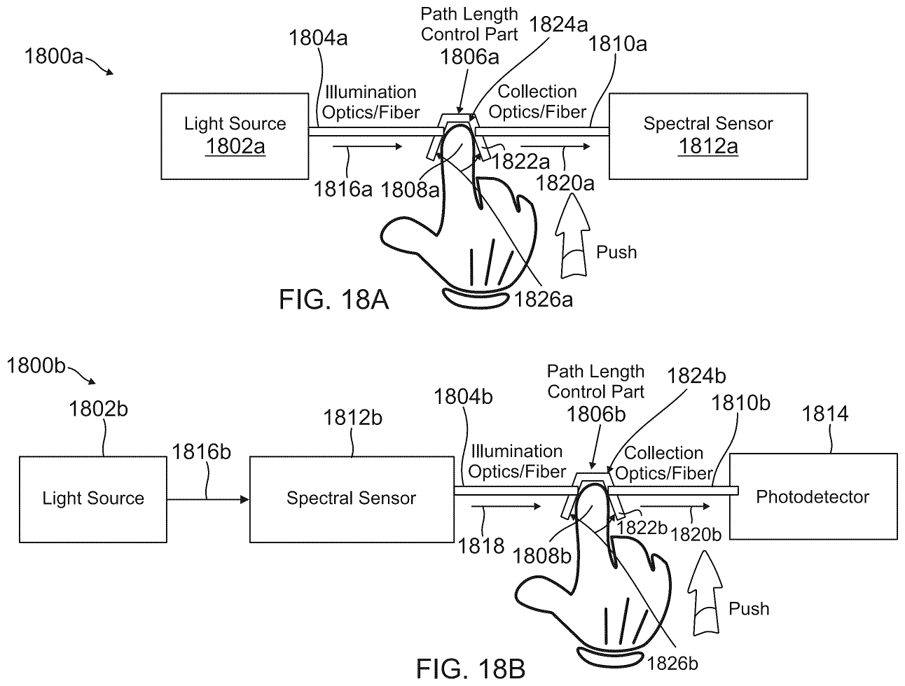

FIGS. 18A and 18B are diagrams illustrating another example of apparatuses configured to control the effective optical path length through a finger according to some aspects. The skin tissue of fingertips may also be thin enough, allowing transmission spectroscopy with adequate path length.

In the example shown in FIG. 18A, an apparatus 1800a configured for non-invasive optical spectroscopy includes a light source 1802a configured to generate input light 1816a. The light source 1802a may include, for example, a laser source, one or more wideband thermal radiation sources, or a quantum source with an array of light emitting devices that cover the wavelength range of interest. The input light 1818a may be directed to illumination optics 1804a configured to receive the input light 1818a and to direct the input light 1816a to skin tissue 1808a (e.g., fingertip) contained within a path length control part 1806a. As shown in FIG. 18A, the skin tissue 1808a may correspond to a portion of a fingertip that can be fit into a mechanical part 1822a of the path length control part 1806a. For example, the mechanical part 1822a may include an opening 1824a within which a subject may insert the fingertip and walls 1826a of the opening 1824a against which the subject may press the fingertip to allow the skin tissue 1808a to tightly fit through the mechanical part 1822a. A distance between the two walls 1826a of the opening 1824a can be configured to control the effective optical path length.

The input light 1816a is transmitted through the skin tissue 1808a, where the light scatters to produce diffusely scattered light 1820a. The path length control part 1806a is configured to control the effective optical path length of the diffusely scattered light 1820a transmitted through the skin tissue 1808a to produce a target effective optical path length of the diffusely scattered light 1820a through the skin tissue 1808a. For example, the path length control part may be configured to compress and hold the skin tissue 1808a in place during analyte measurement. The target effective optical path length may be dependent, for example, on the analyte (e.g., blood biochemical or biomarker) of the skin tissue 1808a under test. For example, the target effective path length may be dependent upon the wavelength of interest for the biomarker.

The diffusely scattered light 1820a output from the skin tissue 1808a is coupled to collection optics 1810a configured to receive the diffusely scattered light 1820a and direct the diffusely scattered light 1820a to a spectral sensor 1812a. The spectral sensor 1812a shown in FIG. 18A may be, for example, a spectrometer including a detector to obtain a spectrum of the analyte under test. The spectrometer 1812a may include, for example, a Fourier Transform infrared (FTIR) spectrometer that exploits light interference and Fourier transform to produce a spectrum of the analyte under test. For example, the spectrometer 1812a may include a Michelson FTIR interferometer in which the spectrum may be retrieved, for example, using a Fourier transform carried out by a processor (not shown). The spectrometer 1812a is not limited to a Michelson FTIR interferometer, and may include any spectrometer type, such as a Fabry-Perot spectrometer, a diffraction grating spectrometer, or other suitable type of spectrometer.

In the example shown in FIG. 18B, an apparatus 1800b configured for non-invasive optical spectroscopy includes a light source 1802b configured to generate input light 1816b. The light source 1802b may include, for example, a laser source, one or more wideband thermal radiation sources, or a quantum source with an array of light emitting devices that cover the wavelength range of interest. The input light 1816b may be directed to a spectral sensor 1812b configured to receive the input light 1816b and to produce modulated light 1818 based on the input light 1816b. For example, the spectral sensor 1812b may be an interferometer (e.g., a Michelson interferometer) or other suitable type of spectral sensor, such as a diffraction element, a Fabry-Perot cavity, a spatial spectral sensor, or a birefringent device.

The modulated light 1818 may be directed to illumination optics 1804a configured to receive the modulated light 1818 and to direct the modulated light 1818 to skin tissue 1808b (e.g., fingertip) contained within a path length control part 1806b. As shown in FIG. 18B, the skin tissue 1808b may correspond to a portion of a fingertip that can be fit into a mechanical part 1822b of the path length control part 1806b. For example, the mechanical part 1822b may include an opening 1824b within which a subject may insert the fingertip and walls 1826b of the opening 1824b against which the subject may press the fingertip to allow the skin tissue 1808b to tightly fit through the mechanical part 1822b. A distance between the two walls 1826b of the opening 1824b can be configured to control the effective optical path length. The modulated light 1818 is transmitted through the skin tissue 1808b, where the light scatters to produce diffusely scattered light 1820b. The path length control part 1806a is configured to control the effective optical path length of the diffusely scattered light 1820b transmitted through the skin tissue 1808b to produce a target effective optical path length of the diffusely scattered light 1820b through the skin tissue 1808b, as described above.

The diffusely scattered light 1820b output from the skin tissue 1808b is coupled to collection optics 1810b configured to receive the diffusely scattered light 1820b and direct the diffusely scattered light 1820b to a detector 1814 (e.g., a photodetector) to obtain a spectrum of the analyte under test. In some examples, the detector 1814 may include a single detector, a detector array, or a multi-pixel detector. In examples in which the spectral sensor 1812b is a FT-IR spectrometer or Fabry-Perot spectrometer, the modulated light 1818 may correspond to interference beams produced over time with an

OPD between beams. The output of the detector 1814 may then correspond to an interferogram, which may be input to a processor (not shown) to retrieve the spectrum. In examples in which the spectral sensor 1812b is a diffraction grating, the modulated light 1818 may correspond to diffracted light across a plurality of wavelengths. The output of the detector 1814 may then correspond to an image representing the light intensity at each wavelength point on the detector, which may be input to the processor (not shown) to retrieve the spectrum.

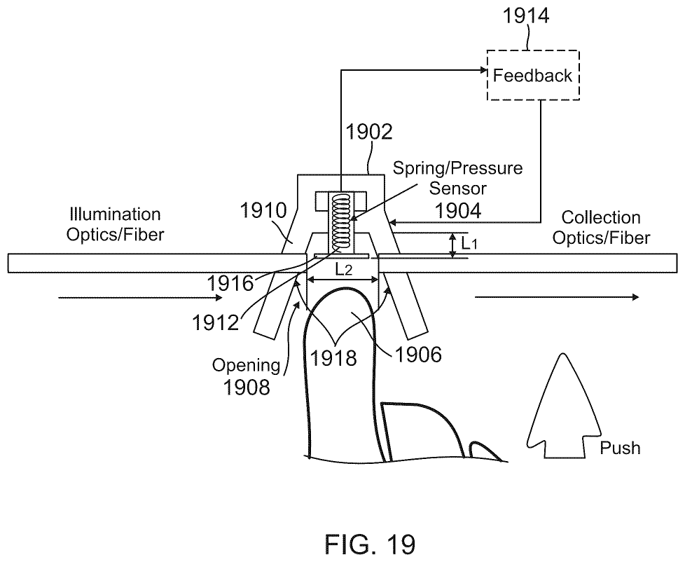

FIG. 19 is a diagram illustrating an example of a path length control part for the tip of a finger according to some aspects. In the example shown in FIG. 19, the path length control part 1902 includes a mechanical part 1910, which includes an opening 1908 and walls 1918 of the opening 1908 against which pressure may be applied by a subject to insert the skin tissue 1906 into the opening 1908. The mechanical part 1910 is thus configured to compress and hold the skin tissue 1906 under test to produce a target effective optical path length through the skin tissue 1906.

The mechanical part 1910 further includes pressure sensor 1904 configured to measure the pressure applied by the subject to the mechanical part 1910. In some examples, the pressure sensor 1904 may include a spring-loaded part 1912. The spring-loaded part 1912 may be configured to measure a distance or displacement (L₁) of the spring pushed by the fingertip. The distance (L₁) may then be used to calculate the effective optical path length (L₂), corresponding to a distance between the two walls 1918 of the opening 1908. The mechanical part 1910 may further including a locking mechanism 1916 configured to lock the fingertip in place when the target effective optical path length is reached. For example, a feedback device 1914 may further be coupled with the mechanical part 1910 and locking mechanism 1916 to calculate the effective optical path length based on the distance (L₁) and to further lock the skin tissue 1906 in place in response to the effective optical path length reaching the optimal optical path length. In some examples, the feedback device 1914 may be further configured to calculate the distance (L₁) based on the pressure applied to the pressure sensor.

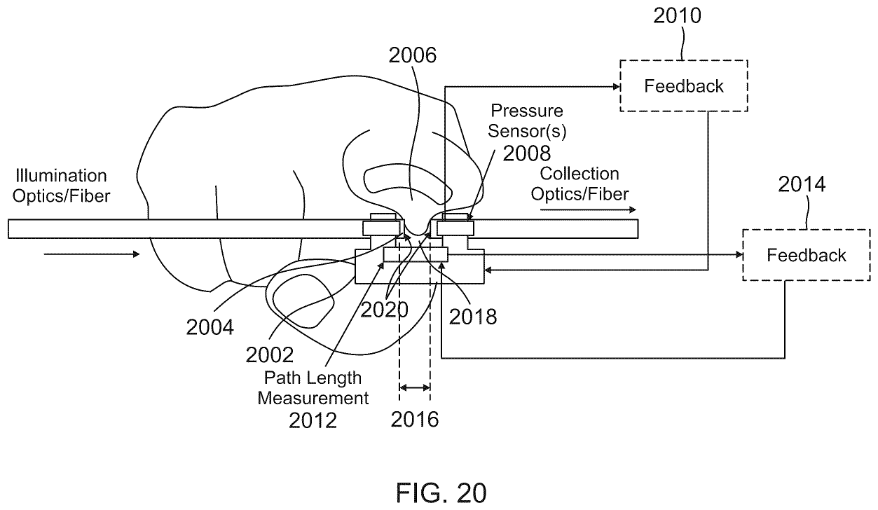

FIG. 20 is a diagram illustrating an example of a path length control part for the bottom of a finger according to some aspects. In the example shown in FIG. 20, the path length control part 2002 includes a mechanical part 2004 configured to hold skin tissue 2006 corresponding to a bottom of a fingertip in place. The mechanical part 2004 is configured to compress and hold the skin tissue 2006 under test to produce a target effective optical path length through the skin tissue 2006. The mechanical part 2004 includes an opening 2018 within which a subject can insert the bottom of the fingertip and walls 2020 of the opening 2018 against which pressure can be applied to secure the skin tissue 2006 of the fingertip. A distance between the two walls 2020 of the opening 2018 can be configured to control the effective optical path length.

The mechanical part 2004 further includes pressure sensor 2008 configured to measure the pressure applied by the mechanical part 2004 to the skin tissue 2006. A pressure feedback device 2010 may further be coupled to the mechanical part 2004 and configured to adjust the mechanical part 2004 based on the pressure applied to the skin tissue 2006. The mechanical part 2004 may further include a path length measurement device 2012 configured to measure a thickness 2016 of the skin tissue 2006 corresponding to the effective optical path length. A path length feedback device 2014 may further be coupled with the mechanical part 2004 to adjust the mechanical part 2004 based on the thickness 2016 to produce (e.g., achieve) a target effective optical path length.

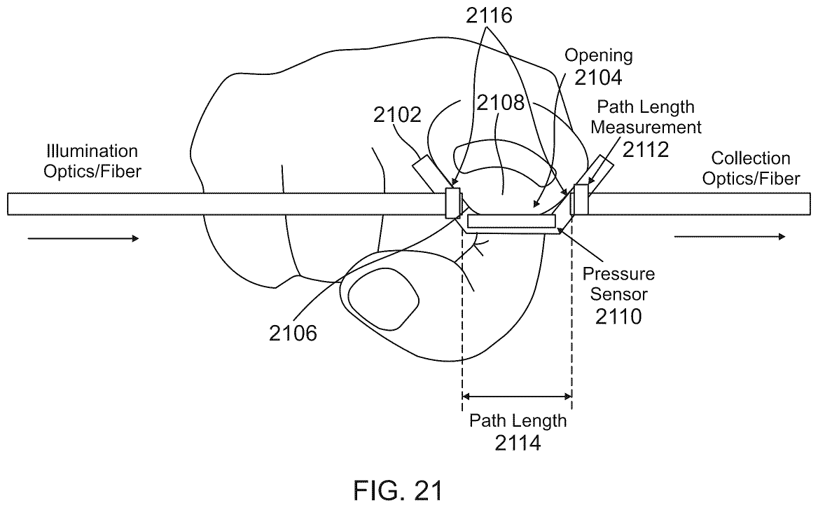

FIG. 21 is a diagram illustrating another example of a path length control part for the bottom of a finger according to some aspects. In the example shown in FIG. 21, a path length control part 2102 includes a mechanical part 2106, which includes an opening 2104 and walls 2116 of the opening configured to receive skin tissue 2108 corresponding to a bottom of a finger (fingertip) and against which pressure may be applied by a subject to insert the skin tissue 2108 into the opening 2104. The mechanical part 2106 is thus configured to compress (e.g., squeeze) and hold the skin tissue 2108 under test to produce a target effective optical path length through the skin tissue 2108. A distance between the two walls 2116 of the opening 2104 can be configured to control the effective optical path length.

The mechanical part 2106 further includes pressure sensor 2110 configured to measure the pressure applied by the mechanical part 2106 to the skin tissue 2108. The mechanical part 2106 may further include a path length measurement device 2112 configured to measure an effective optical path length 2114. One or more feedback devices (not shown) may further be included to adjust the mechanical part 2106 or to provide feedback to a user (e.g., to apply more pressure) based on the measured pressure and/or measured effective optical path length to produce the target effective optical path length through the skin tissue 2108.

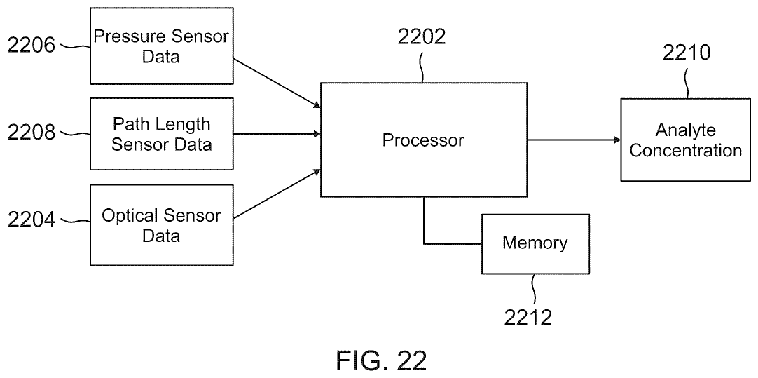

FIG. 22 is a diagram illustrating an example of spectrum processing according to some aspects. In the example shown in FIG. 22, a processor 2202 (e.g., the processor 214a or 214b shown in FIG. 2 or other processor) is configured to receive and process optical sensor data 2204 to calculate a concentration of an analyte under test (e.g., an analyte concentration 2210) using, for example, one or more algorithms maintained in a memory 2212 coupled to the processor. The optical sensor data 2204 may correspond, for example, to a spectrum (e.g., obtained from optical transmission spectroscopy of skin tissue of a subject), an interferogram from which the spectrum may be obtained by the processor 2202, or other suitable optical sensor data.

Examples of processors 2202 include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described throughout this disclosure. The memory 2212 may include, for example, a non-transitory computer-readable medium. A non-transitory computer-readable medium includes, by way of example, a magnetic storage device (e.g., hard disk, floppy disk, magnetic strip), an optical disk (e.g., a compact disc (CD) or a digital versatile disc (DVD)), a smart card, a flash memory device (e.g., a card, a stick, or a key drive), a random access memory (RAM), a read only memory (ROM), a programmable ROM (PROM), an erasable PROM (EPROM), an electrically erasable PROM (EEPROM), a register, a removable disk, and any other suitable medium for storing software and/or instructions (e.g., an algorithm for calculating the analyte concentration 2210) that may be accessed and read by a processor 2202.

Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. The software, when executed by the processor 2202, causes the processor 2202 to perform the various functions described herein. The memory 2212 may also be used for storing data that is utilized by the processor 2202 when executing software.

In some examples, the processor 2202 may further be configured to receive pressure sensor data 2206 and/or path length sensor data 2208. The processor 2202 may be configured to calculate the analyte concentration 2210 based on the optical sensor data 2204, and at least one of the pressure sensor data 2206 or the path length sensor data 2208. The pressure sensor data 2206 may be produced, for example, by one of the pressure sensors shown in FIGS. 16B, 19, 20, and/or 21. The path length sensor data 2208 may be produced, for example, by one of the path length measurement devices shown in FIGS. 16C, 19, 20, and/or 21.

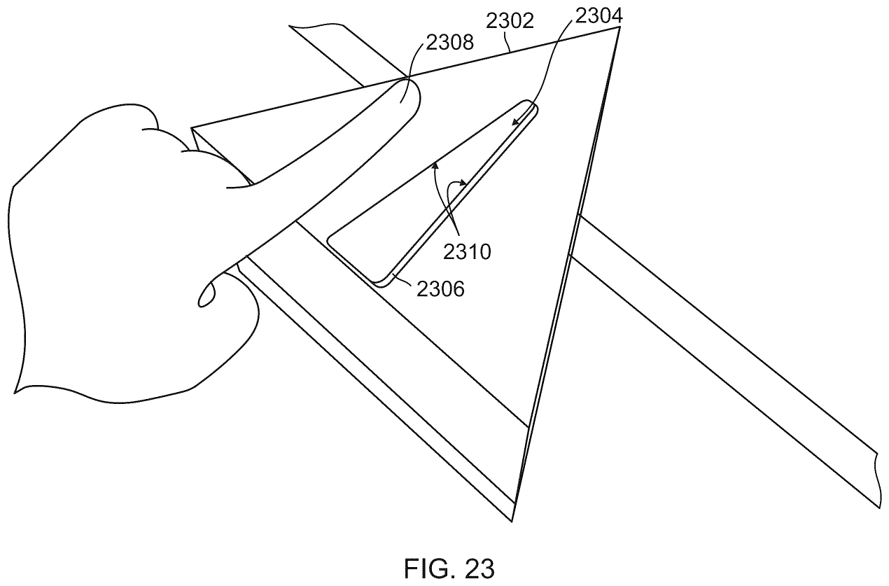

FIG. 23 is a diagram illustrating another example of a path length control part for the bottom of a finger according to some aspects. In the example shown in FIG. 23, the path length control part 2302 includes a mechanical part 2306, which includes an opening 2304 configured to receive skin tissue 2308 corresponding to a bottom of a finger (fingertip) and walls 2310 of the opening 2304 against which pressure may be applied by a subject to insert the skin tissue 2308 into the opening 2304. The mechanical part 2306 is configured to compress and hold the skin tissue 2308 under test to produce a target effective optical path length through the skin tissue 2308.

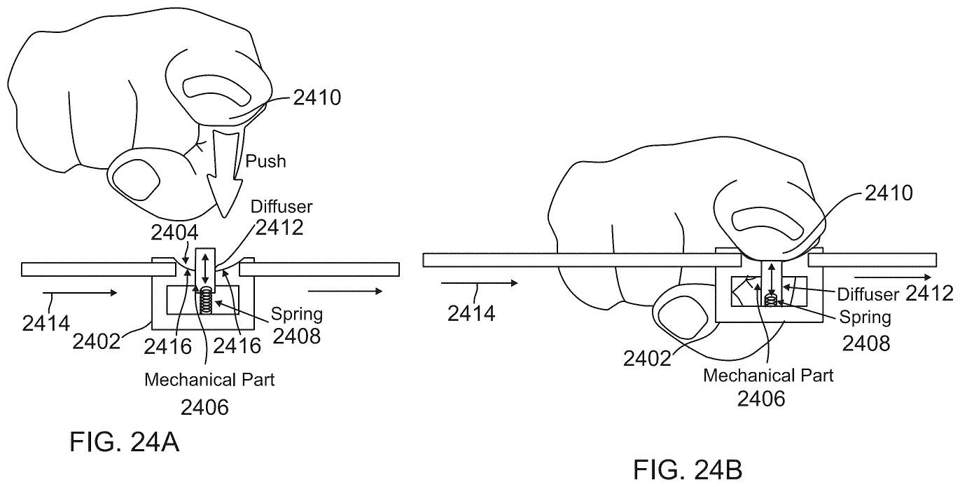

FIGS. 24A and 24B are diagrams illustrating an example of a path length control part further configured to enable a background measurement according to some aspects. In the example shown in FIGS. 24A and 24B, the path length control part 2402 includes a mechanical part 2406, which includes an opening 2404 configured to receive skin tissue 2410 corresponding to a bottom of a finger (fingertip) and walls 2416 of the opening 2404 against which pressure may be applied by a subject to insert the skin tissue 2410 into the opening 2404. The mechanical part 2406 is configured to compress and hold the skin tissue 2410 under test to produce a target effective optical path length through the skin tissue 2410.

The mechanical part 2406 includes a spring 2408 coupled to a diffuser 2412 forming a spring-loaded moveable diffuser. The spring-loaded diffuser 2408/2412 may be configured to be in a first position in a light path 2414 of the apparatus, as shown in FIG. 24A, prior to pressure being applied to the spring-loaded diffuser 2408/2412 of the mechanical part 2406 to obtain a background spectrum (e.g., for calibration of the apparatus). As shown in FIG. 24B, after pressure is applied to the spring-loaded diffuser 2408/2412 by the bottom of the finger of the subject to push the spring-loaded diffuser 2408/2412 down, the spring-loaded diffuser 2408/2412 is moved to a second position out of the light path 2414 to enable the skin tissue 2410 to move into the light path 2414 to obtain a spectrum of an analyte under test of the subject. In some examples, a pressure sensor and/or path length measurement device with feedback may further be included to adjust the second position of the spring-loaded diffuser 2408/2412 to produce a target effective optical path length. In some examples, a locking mechanism may also be included to lock the spring-loaded diffuser 2408/2412 in place in response to achieving the target effective optical path length.

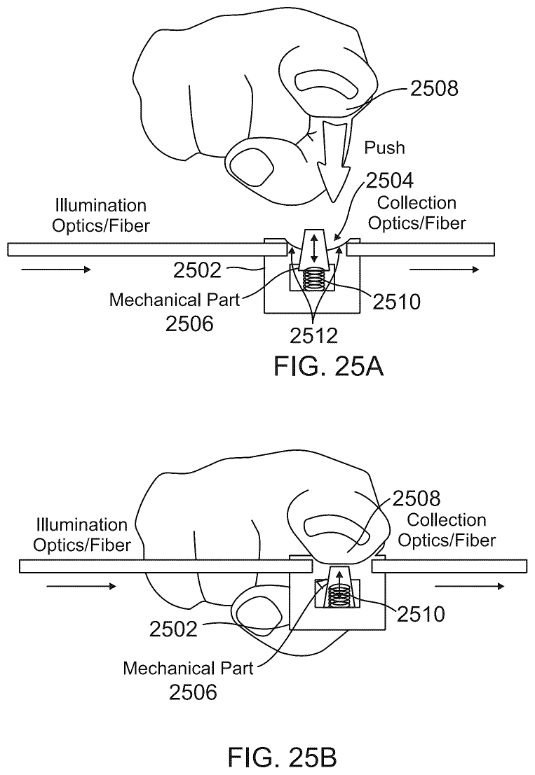

FIGS. 25A and 25B are diagrams illustrating another example of a path length control part for the bottom of a finger according to some aspects. In the example shown in FIGS. 25A and 25B, the path length control part 2502 includes a mechanical part 2506, which includes an opening 2504 configured to receive skin tissue 2508 corresponding to a bottom of a finger (fingertip) and walls 2512 of the opening 2504 against which pressure may be applied by a subject to insert the skin tissue 2508 into the opening 2504. The mechanical part 2506 is configured to compress and hold the skin tissue 2508 under test to produce a target effective optical path length through the skin tissue 2508.

The mechanical part 2506 includes a spring-loaded part 2510 configured to be displaced in response to pressure applied thereto. The spring-loaded part 2510 may be configured to automatically lock into place after a sufficient displacement thereof, indicating that pressure applied by the bottom of the finger (skin tissue 2508) has reached a desired amount corresponding to a target effective optical path length through the skin tissue 2508. The mechanical part 2406 may then hold the skin tissue 2508 in place to obtain a spectrum of the analyte under test of the skin tissue 2508.

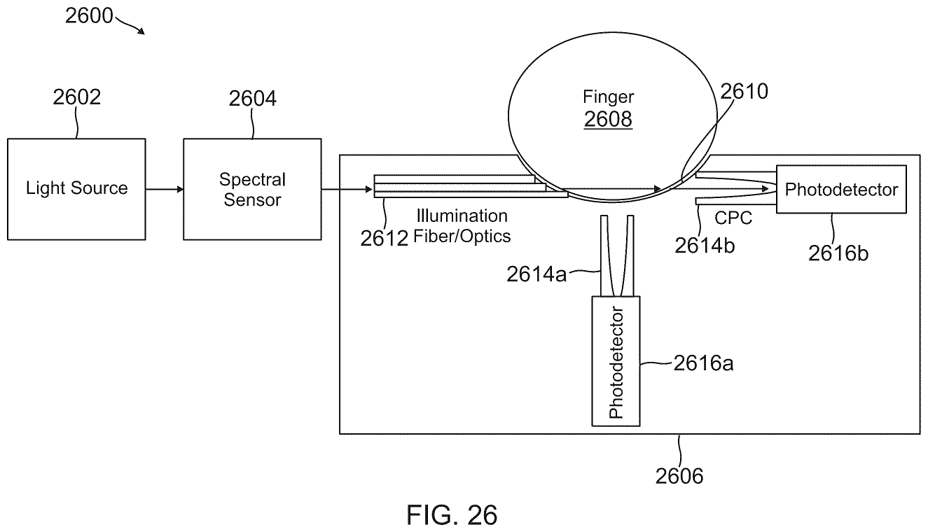

FIG. 26 is a diagram illustrating an example of an apparatus including multiple detectors to measure the diffusely scattered light from the bottom and sides of the finger according to some aspects. In the example shown in FIG. 26, an apparatus 2600 configured for non-invasive optical spectroscopy includes a light source 2602 configured to generate input light and to direct the input light to a spectral sensor 2604. The spectral sensor 2604 is configured to produce modulated light based on the input light and to direct the modulated light to a path length control part 2606.

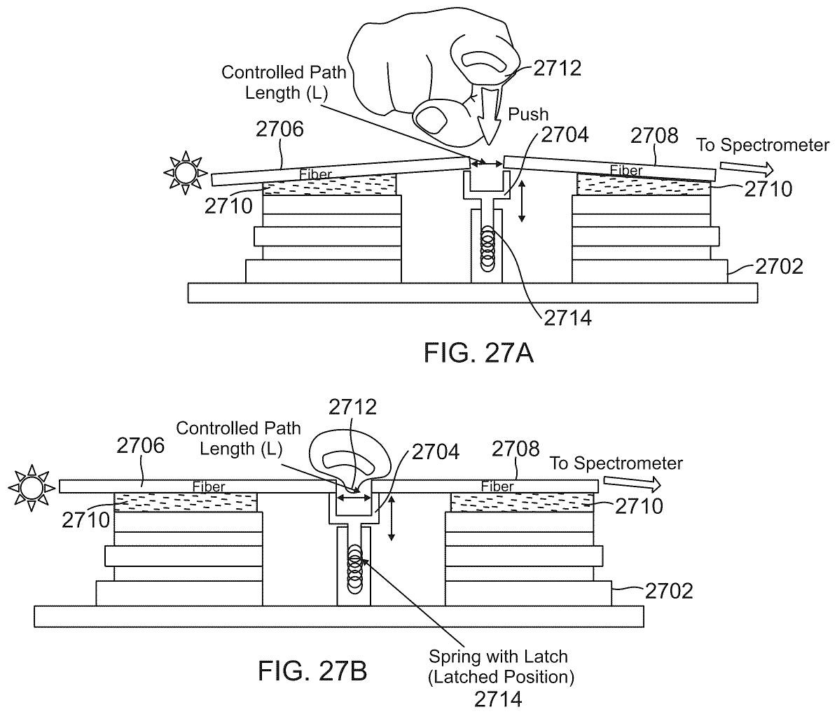

The path length control part 2606 may include illumination optics 2612 configured to receive the modulated light and to direct the modulated light to skin tissue 2608 (e.g., bottom of a finger) contained within a mechanical part 2610 of the path length control part 2606. The mechanical part 2610 is configured to compress and hold the skin tissue 2608 to produce a target effective optical path length through the skin tissue 2608. The modulated light is the transmitted through the skin tissue 2608, where the light scatters to produce diffusely scattered light. The path length control part further includes collection optics 2614a and 2614b (e.g., CPCs) configured to receive the diffusely scattered light from multiple surfaces of the skin tissue 2608 (e.g., side and bottom) and to direct the diffusely scattered light towards respective detectors 2616a and 2616b (e.g., photodetectors). In the example shown in FIG. 27, two detectors 2616a and 2616b are shown. However, it should be understood that additional detectors (e.g., three or more) may be utilized to capture the diffusely scattered light from different sides and/or the bottom of the finger.

FIGS. 27A and 27B are diagrams illustrating another example of a path length control part according to some aspects. In the example shown in FIGS. 27A and 27B, the path length control part 2702 includes a mechanical part 2704 against which pressure may be applied by a subject to insert skin tissue 2712 corresponding to a bottom of a finger (fingertip). The mechanical part 2704 is configured to compress and hold the skin tissue 2712 under test to produce a target effective optical path length through the skin tissue 2712.

The path length control part 2702 further includes a portion of illumination optics 2706 (e.g., optical fiber(s)) and collection optics 2708 (e.g., optical fiber(s)) fixed (mounted) onto a moveable tilting component 2710. The mechanical part 2704 further includes respective ends of the illumination and collection optics 2706 and 2708 that may be coupled to a spring with latch 2714 in response to pressure applied to the mechanical part 2704 by the bottom of the finger. For example, the moveable tilting component 2710 may be configured to tilt the illumination optics 2706 and the collection optics 2708 between a first position, as shown in FIG. 27A, at an angle (θ) from an optical axis of the apparatus and a second position, as shown in FIG. 27B, that is in-plane with the optical axis of the apparatus. When the subject applies force to the illumination optics 2706 and the collection optics 2708, the illumination optics 2706 and collection optics 2708 are configured to tilt or move from the first position to the second position.

In the second position, as shown in FIG. 27B, the spring with latch 2714 snaps in place to fix the illumination optics 2706 and the collection optics 2708 in the second position. In addition, the mechanical part 2704 is configured to compress and hold the skin tissue 2712 of the bottom of the finger in place to obtain a spectrum of an analyte of the skin tissue 2712. In some examples, the spring with latch 2714 may be spring-loaded to enable the illumination and collection optics 2706 and 2708 to return to the first position when the pressure is removed by the subject.

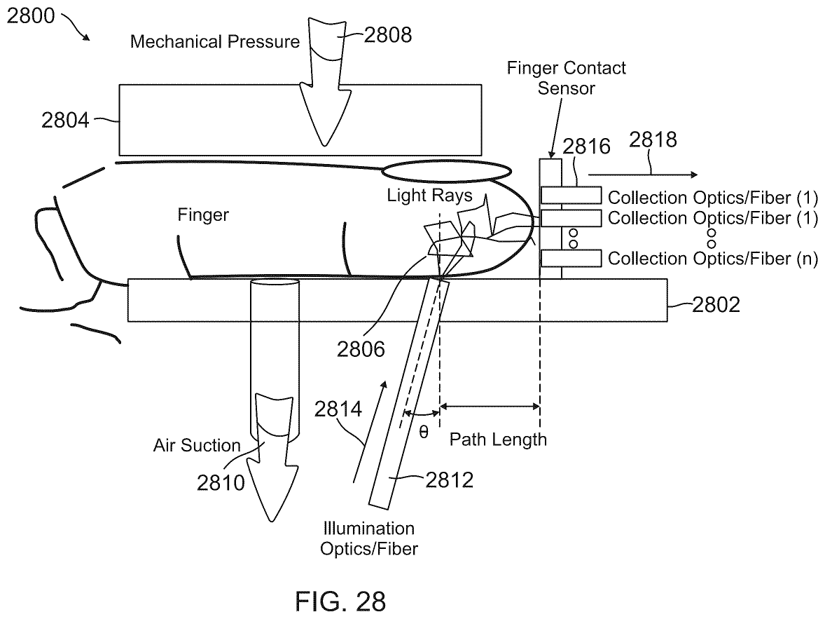

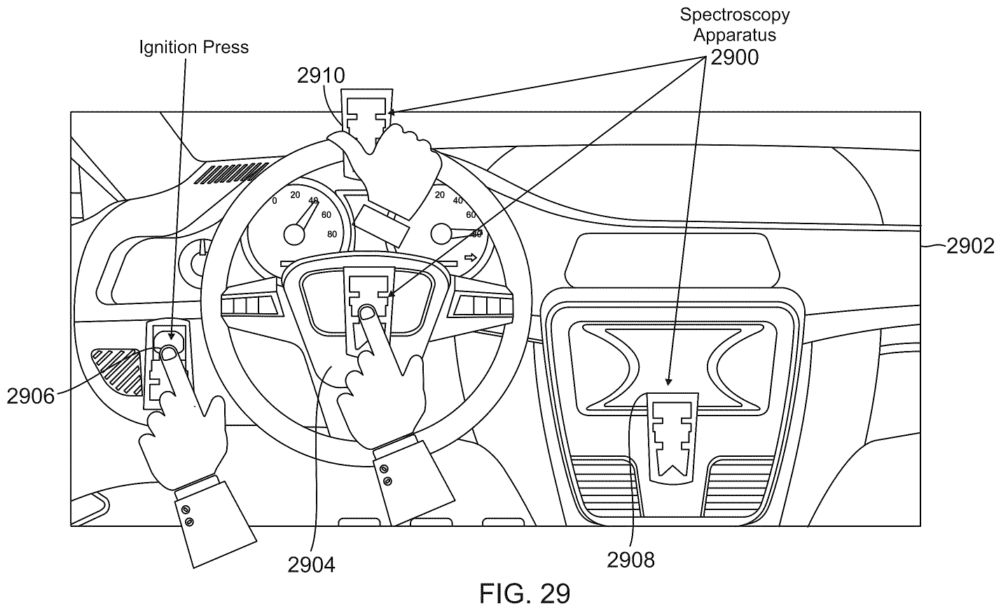

FIG. 28 is a diagram illustrating an example of an apparatus configured to control the effective optical path length through skin tissue using oblique illumination according to some aspects. In the example shown in FIG. 28, the apparatus 2800 includes a path length control part 2802 including a mechanical part 2804 configured to receive skin tissue 2806 corresponding to a finger of a subject. The mechanical part 2804 may be configured to apply mechanical pressure 2808 and suction pressure 2810 to the skin tissue 2806 to compress and hold the skin tissue 2806 in place. The apparatus 2800 further includes illumination optics 2812 configured to direct input light 2814 (or modulated light) to the skin tissue 2806 at the bottom of the finger at an oblique angle (0) for diffused transmission of the input light 2814 (or modulated light) through the skin tissue 2806 to produce diffusely scattered light 2818. The apparatus 2800 further includes collection optics 2816 configured to receive the diffusely scattered light 2818 from the skin tissue 2806. The mechanical and suction pressure applied to the skin tissue 2806 enabled repeatable results with control over the effective optical path length.

FIG. 29 is a diagram illustrating examples of integration of the apparatus into a vehicle according to some aspects. In the example shown in FIG. 29, the apparatus 2900 for non-invasive blood biochemistry optical spectroscopy measurement may be integrated into a component of a vehicle 2902. For example, the apparatus 2900 may be integrated into a steering wheel 2904, an ignition press button 2906, a car console 2908, or a dashboard 2910 of the vehicle 2902. The apparatus 2900 may be configured to measure a physiological parameter (e.g., blood alcohol concentration or glucose level) or to serve as an interlock for the vehicle if the physiological parameter is outside of a certain range. For example, if the driver's blood alcohol concentration or glucose level is above a threshold value, the vehicle may not start or may stop working.

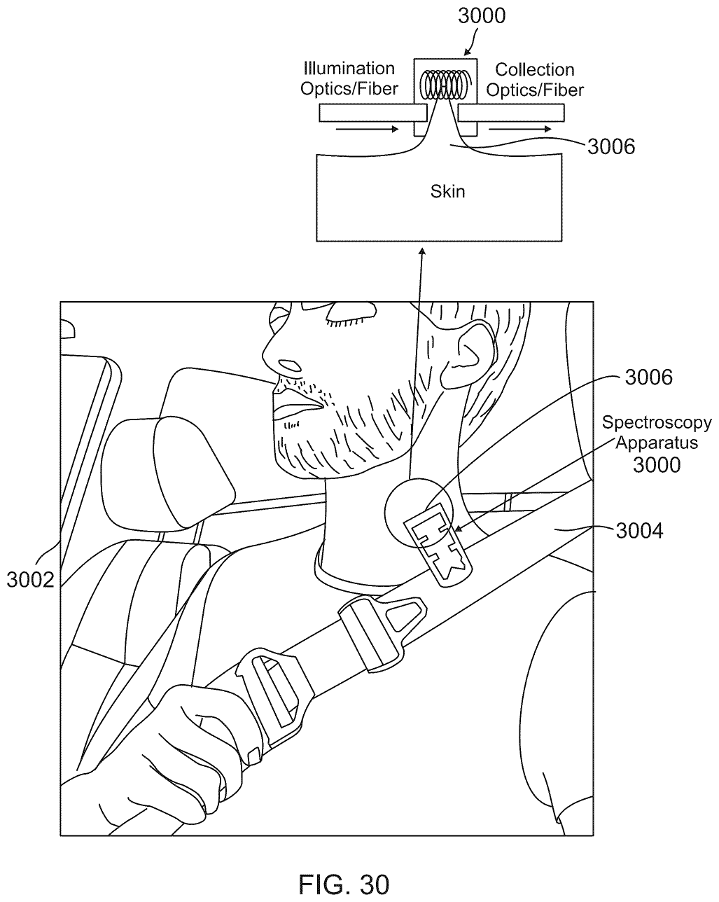

FIG. 30 is a diagram illustrating another example integration of the apparatus into a vehicle according to some aspects. In the example shown in FIG. 30, the apparatus 3000 for non-invasive blood biochemistry optical spectroscopy measurement may be integrated into a seatbelt 3004 of a vehicle 3002. The apparatus 3000 may clip to the driver's neck 3006 to monitor a physiological parameter of the driver and control vehicle operation based on the value of that physiological parameter.

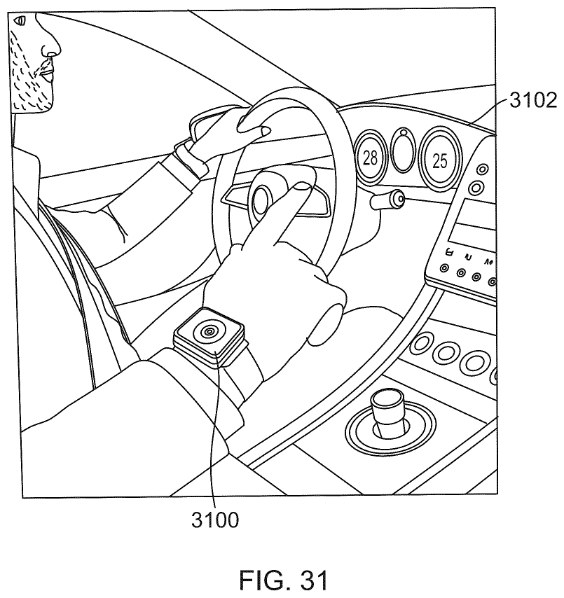

FIG. 31 is a diagram illustrating another example integration of the apparatus into a vehicle according to some aspects. In the example shown in FIG. 31, the apparatus 3100 may be a wearable device connected to the vehicle 3102 via a wired or wireless connection. The apparatus 3100 may be configured to monitor a physiological parameter of the driver and control vehicle operation based on the value of that physiological parameter.

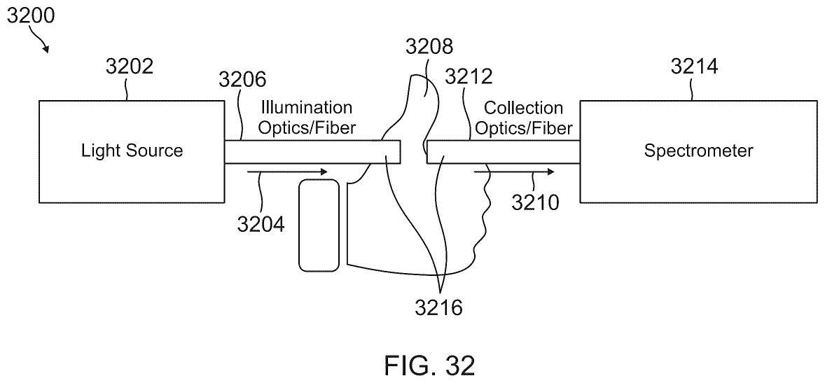

FIG. 32 is a diagram illustrating another example of an apparatus configured to control the effective optical path length through skin tissue according to some aspects. In the example shown in FIG. 32, the apparatus 3200 configured for non-invasive optical spectroscopy includes a light source 3202 configured to generate input light 3204 (e.g., multi-wavelength light). The input light 3204 may be directed to skin tissue 3208 (e.g., interdigital web) via illumination optics 3206. The input light 3206 is transmitted through the skin tissue 3208, where the light scatters to produce diffusely scattered light 3210 that is directed to a spectrometer 3214 via collection optics 3212. The illumination optics 3206 and collection optics 3212 collectively form a path length control part 3216 configured to control the effective optical path length of the diffusely scattered light 3210 transmitted non-invasively through the skin tissue 3208 to produce a target effective optical path length of the diffusely scattered light 3210 through the skin tissue 3208. For example, the path length control part may be configured to compress and hold the skin tissue 3208 in place during analyte measurement. In some examples, micro-positioners (not shown) may be coupled to the illumination and collection optics 3206 and 3212 to allow inserting the skin tissue 3208 into a groove between the illumination optics 3206 and the collection optics 3212 and adjusting the path length or pressure of the illumination and collection optics 3206 and 3212, so that measurements can be made at a constant pressure and a constant path length. A similar configuration using the illumination and collection optics as the path length control part may be utilized to measure through other types of skin tissue, such as a tip of a finger, the bottom of a fingertip, an earlobe, a wrist, a nose, a portion of a neck, etc.

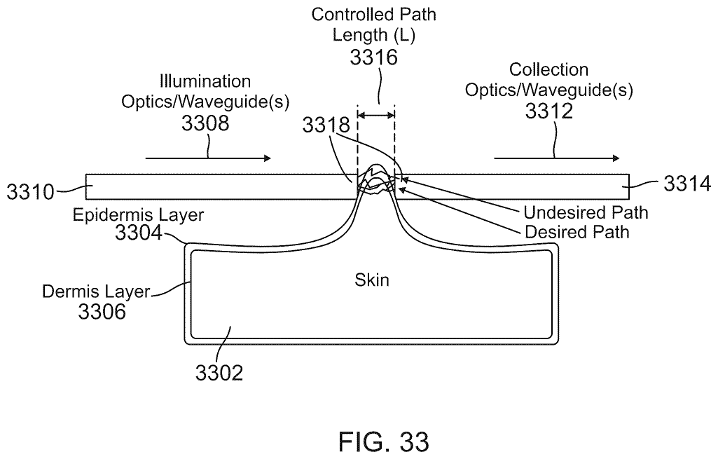

FIG. 33 is a diagram illustrating another example of illumination and collection optics according to some aspects. In the example shown in FIG. 33, illumination optics 3310 are optically coupled to direct input light (or modulated light) 3308 to skin tissue 3302. The light (input or modulated) 3308 is transmitted through the skin tissue 3302 to produce diffusely scattered light 3312 that is collected by collection optics 3314. The input/modulated light 3308 is transmitted through the skin tissue 3302 with a target effective optical path length (L) 3316 produced by a path length control part 3318 formed by the illumination optics 3310 and the collection optics 3314 (e.g., micro-positioners configured to control the illumination and collection optics 3310 and 3314 to allow for insertion of the skin tissue 3302 into a groove therebetween and to adjust the effective path length therebetween). For example, the skin tissue 3302 may be compressed and held at a thickness selected to produce a target effective path length. In addition, as shown in FIG. 33, the illumination and/or collection optics 3310/3314 may be configured to minimize the amount of light passing through an epidermis layer 3304 of the skin tissue 3302 without also passing through a dermis layer 3306 of the skin tissue 3302. In some examples, the dermis layer 3306 may contain most of the analytes that may be detected non-invasively through the skin tissue 3302.

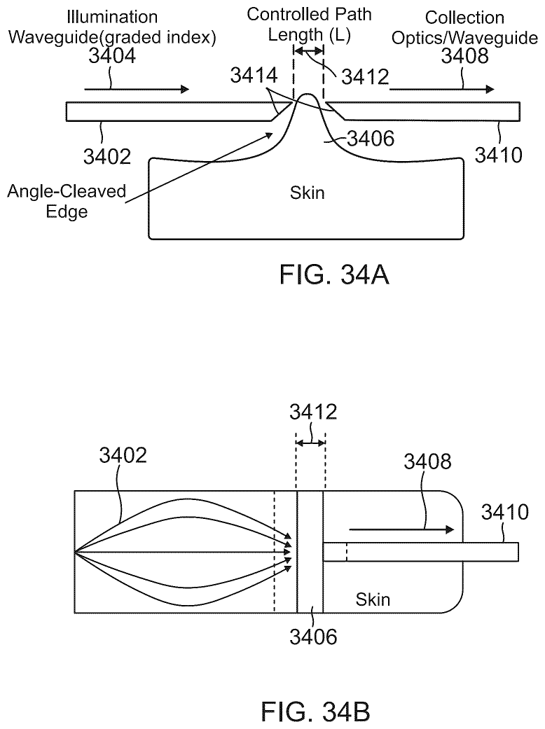

FIGS. 34A and 34B are diagrams illustrating another example of illumination and collection optics according to some aspects. FIG. 34A is a side view, whereas FIG. 34B is a top view. In the example shown in FIGS. 34A and 34B, illumination optics 3402, which may include a plurality of waveguides, are optically coupled to direct input light (or modulated light) 3404 to skin tissue 3406. The light (input or modulated) 3404 is transmitted through the skin tissue 3406 to produce diffusely scattered light 3408 that is collected by collection optics 3410. The input/modulated light 3404 is transmitted through the skin tissue 3406 with a target effective optical path length (L) 3412 produced by a path length control part 3414 formed by the illumination optics 3402 and the collection optics 3410 (e.g., using micro-positioners). For example, the skin tissue 3406 may be compressed and held at a thickness selected to produce a target effective path length. As further shown in the example of FIG. 34A, the illumination optics 3402 may include graded-index illumination waveguides configured to focus the input light 3404 onto the skin tissue 3406 in the lateral direction. In addition, respective tips of the illumination waveguides 3402 and/or collection waveguide 3410 may be cleaved at an angle to enable the skin tissue 3406 to fit between the illumination optics 3402 and collection optics 3410 more easily.

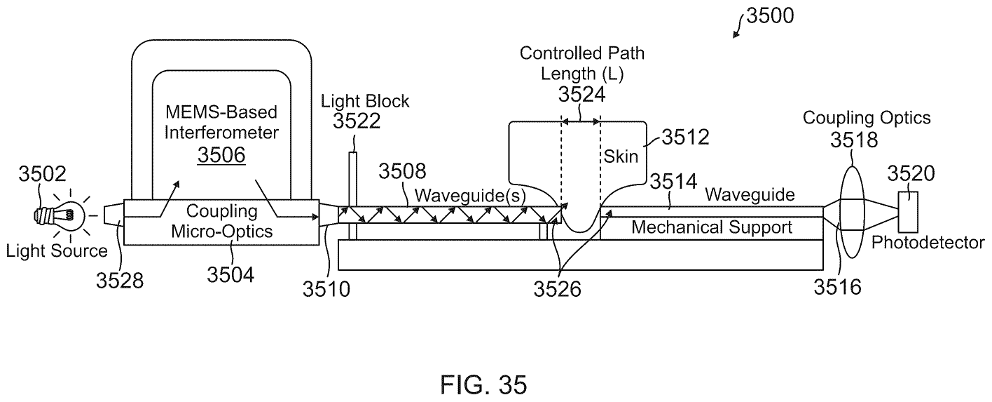

FIG. 35 is a diagram illustrating another example of an apparatus configured to control the effective optical path length through skin tissue according to some aspects. In the example shown in FIG. 35, the apparatus 3500 configured for non-invasive optical spectroscopy includes a light source 3502 configured to generate input light 3528 (e.g., multi-wavelength light). The input light 3528 may be directed to a light modulator (e.g., a MEMS-based interferometer) 3506 to produce modulated light 3510 that is directed to skin tissue 3512 via illumination optics (e.g., one or more waveguides) 3508. An integrated micro-optical component 3504 may be configured to couple the input light 3528 into the light modulator 3506 and the modulated light 3510 out of the light modulator 3506. A light block component 3522 may further be integrated onto the surface of the illumination waveguide 3508 to prevent stray light from reflecting back towards the light modulator 3506.

The modulated light 3510 is transmitted through the skin tissue 3512, where the light scatters to produce diffusely scattered light 3516 that is directed to a detector 3520 (e.g., a photodetector) via collection optics 3514 (e.g., a waveguide) and coupling optics 3518. The illumination optics 3508 and collection optics 3514 collectively form a path length control part 3526 configured to control the effective optical path length of the diffusely scattered light 3516 transmitted non-invasively through the skin tissue 3512 to produce a target effective optical path length of the diffusely scattered light 3516 through the skin tissue 3512. For example, the path length control part 3526 may be configured to compress and hold the skin tissue 3512 in place during analyte measurement. The apparatus 3500 shown in FIG. 35 aids in reducing skin heating by filtering the light and allowing the light to undergo losses in the light modulator 3506 before reaching the skin tissue 3512.

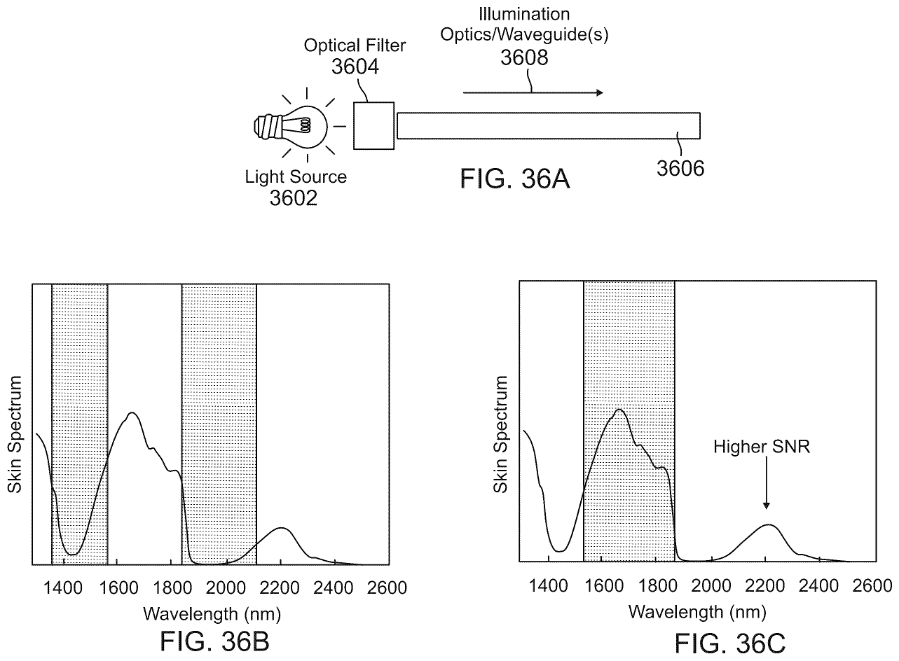

FIGS. 36A-36C illustrate an example of filtering input light according to some aspects. As shown in FIG. 36A, an optical filter 3604 (e.g., spectral filter) may be inserted into the illumination path after the light source 3602 and prior to the illumination optics 3606 (e.g., illumination waveguide(s)) to filter out wavelength ranges with high water absorption. Therefore, the optical filter 3604 can be configured to reduce the amount of input light 3608 (or modulated light) reaching the skin tissue, thus reducing the heating of the skin tissue. In addition, as shown in FIGS. 36B and 36C, the optical filter 3604 can further be configured to decrease the power of wavelength ranges that have significantly more signal than required for detection of the analyte. By reducing the noise in the detector (e.g., by decreasing the power for wavelength ranges that have more signal than required for detection), the signal-to-noise ratio (SNR) for other wavelength ranges that have smaller light intensity, and which may contain important analyte absorption peaks, may be enhanced.

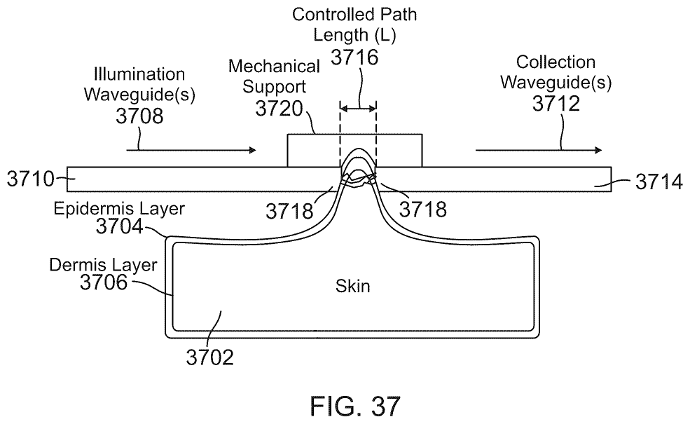

FIG. 37 is a diagram illustrating another example of illumination and collection optics according to some aspects. In the example shown in FIG. 37, illumination optics 3710 are optically coupled to direct input light (or modulated light) 3708 to skin tissue 3702. The light (input or modulated) 3708 is transmitted through the skin tissue 3702 to produce diffusely scattered light 3712 that is collected by collection optics 3714. The input/modulated light 3708 is transmitted through the skin tissue 3702 with a target effective optical path length (L) 3716 produced by a path length control part 3718 formed by the illumination optics 3710 and the collection optics 3714. For example, the skin tissue 3702 may be compressed and held at a thickness selected to produce a target effective path length.

In addition, as shown in FIG. 37, the illumination and/or collection optics 3710/3714 may be configured to minimize the amount of light passing through an epidermis layer 3704 of the skin tissue 3702 without also passing through a dermis layer 3706 of the skin tissue 3702. A mechanical support part 3720 may further be included to support the illumination optics 3710 and/or collection optics 3714 with a curve that enables the skin tissue 3702 to penetrate more between the illumination optics 3710 and collection optics 3714, which may allow more light to pass through the dermis layer 3706. In some examples, the mechanical support part may be made of plastic of polyetheretherketone (PEEK) material.

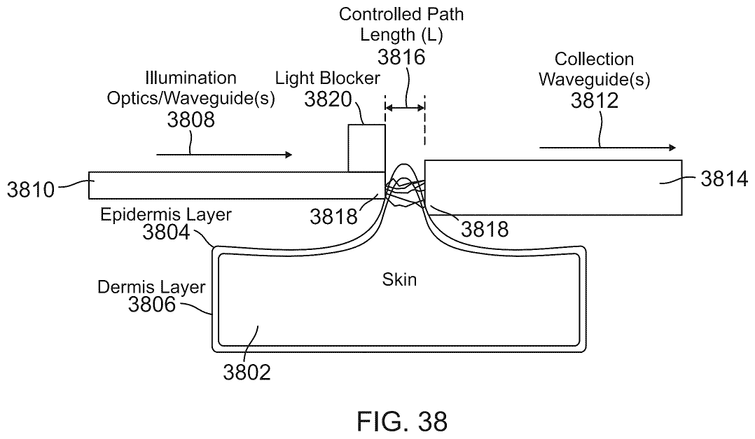

FIG. 38 is a diagram illustrating another example of illumination and collection optics according to some aspects. In the example shown in FIG. 38, illumination optics 3810 are optically coupled to direct input light (or modulated light) 3808 to skin tissue 3802. The light (input or modulated) 3808 is transmitted through the skin tissue 3802 to produce diffusely scattered light 3812 that is collected by collection optics 3814. The input/modulated light 3808 is transmitted through the skin tissue 3802 with a target effective optical path length (L) 3816 produced by a path length control part 3818 formed by the illumination optics 3810 and the collection optics 3814. For example, the skin tissue 3802 may be compressed and held at a thickness selected to produce a target effective path length.

In addition, as shown in FIG. 38, the illumination and/or collection optics 3810/3814 may be configured to minimize the amount of light passing through an epidermis layer 3804 of the skin tissue 3802 without also passing through a dermis layer 3806 of the skin tissue 3802. As shown in FIG. 38, the cross-sectional area of the collection optics (collection waveguide) 3814 may be larger than that of the illumination optics (illumination waveguide(s)) 3810, which may allow more light to pass through the dermis layer 3806. In this example, a light blocker 3820 may be inserted onto the illumination optics 3810 to block light from direct coupling from the illumination optics 3810 to the collection optics 3814 without first passing through the skin tissue 3802.

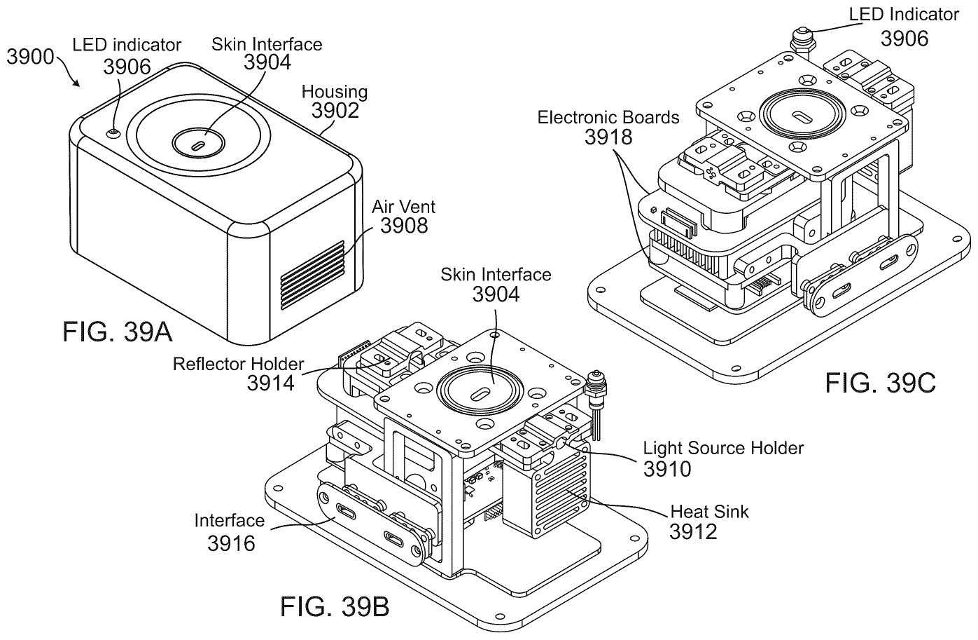

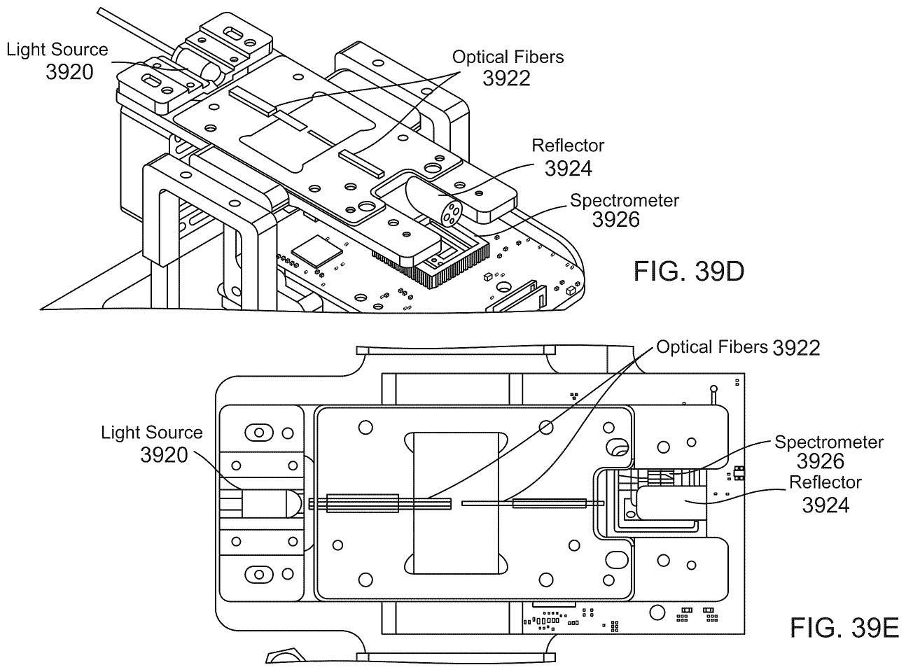

FIGS. 39A-39E are diagrams illustrating another example of an apparatus configured to control the effective optical path length through skin tissue according to some aspects. In the example shown in FIG. 39A, the apparatus 3900 includes a housing 3902 including a skin interface 3904 configured to receive skin tissue inserted into the skin interface 3904, an indicator 3906 (e.g., an LED indicator) configured to indicate when the apparatus is in operation, and an air vent 3908 configured to dissipate heat from the apparatus 3900. In addition, as shown in FIG. 39B, inside the housing 3902, the apparatus 3900 includes a light source holder 3910 configured to hold a light source, a reflector holder 3914 configured to hold a reflector (e.g., optical component configured to reflect diffusely scattered light towards a spectrometer), a heat sink 3912 configured to further dissipate heat from the apparatus 3900, and an optional USB interface 3916.

As further shown in FIG. 39C, the apparatus 3900 may further include one or more electronic boards 3918 configured to support and control components of the apparatus and process data. In some examples, one or more electronic boards 3918 may include a controller configured to control one or more components of the apparatus, and a processor configured to process data (e.g., sensor data and/or a spectral data).

FIGS. 39D and 39E illustrate various optical components of the apparatus 3900, including a light source 3920 (e.g., which may be included in the light source holder 3910), optical fibers 3922 (e.g., illumination and collection waveguides), a reflector 3924 (e.g., which may be included in the reflector holder 3914) and a spectrometer 3926. In the example shown in FIGS. 39D and 39E, the optical fibers 3922 include a plurality of illumination waveguides and a single collection waveguide.

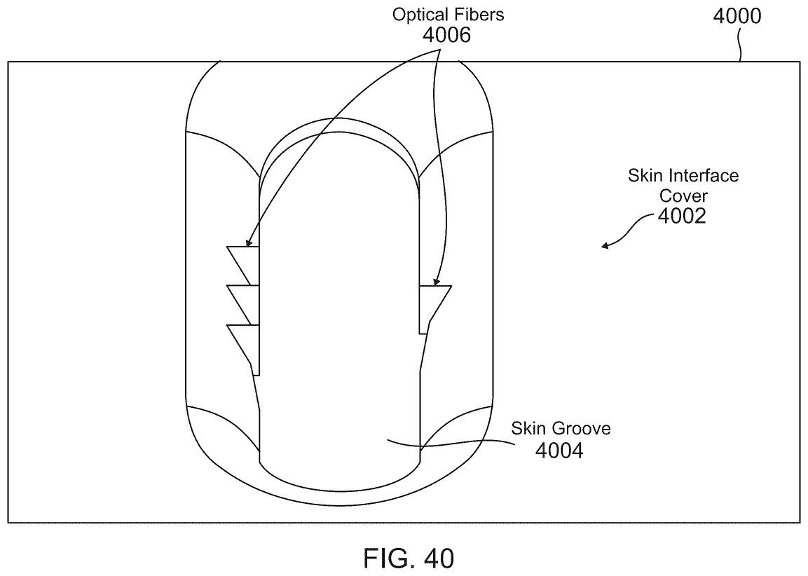

FIG. 40 is a diagram illustrating an example of a skin interface of an apparatus configured to control the effective optical path length through skin tissue according to some aspects. The skin interface 4000 includes a skin interface cover 4002 and a skin groove 4004 within the skin interface cover 4002. The skin groove 4004 is configured to receive skin tissue and is positioned between illumination/collection optical fibers 4006. As shown in the example of FIG. 40, the optical fibers 4006 includes a plurality of illumination waveguides and a single collection waveguide. The effective path length through the skin tissue may be controlled by a path length control part (not specifically shown) coupled to the skin groove 4004. For example, the path length control part may be implemented using the optical fibers 4006 (e.g., using micro-positioners to control the position of the optical fibers 4006 with respect to the inserted skin tissue).

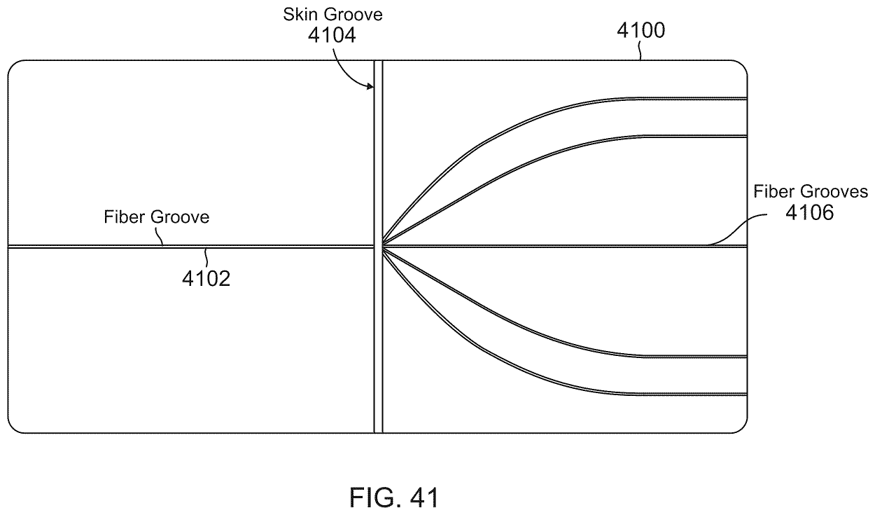

FIG. 41 is a diagram illustrating another example of a skin interface of an apparatus configured to control the effective optical path length through skin tissue according to some aspects. The skin interface 4100 includes a skin groove 4104 configured to receive skin tissue. The skin groove 4104 is positioned between illumination waveguides inserted into illumination fiber grooves 4106 and a collection waveguide inserted into a collection fiber groove 4102. As shown in the example of FIG. 41, the illumination fiber grooves 4106 are configured and positioned with respect to one another to enable each of the illumination waveguides to illuminate the skin tissue inserted into the skin groove 4104 at different angles on the skin.

Within the present disclosure, the word “exemplary” is used to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage or mode of operation. The term “coupled” is used herein to refer to the direct or indirect coupling between two objects. For example, if object A physically touches object B, and object B touches object C, then objects A and C may still be considered coupled to one another-even if they do not directly physically touch each other. For instance, a first object may be coupled to a second object even though the first object is never directly physically in contact with the second object. The terms “circuit” and “circuitry” are used broadly, and intended to include both hardware implementations of electrical devices and conductors that, when connected and configured, enable the performance of the functions described in the present disclosure, without limitation as to the type of electronic circuits, as well as software implementations of information and instructions that, when executed by a processor, enable the performance of the functions described in the present disclosure.

One or more of the components, steps, features and/or functions illustrated in FIGS. 1-41 may be rearranged and/or combined into a single component, step, feature or function or embodied in several components, steps, or functions. Additional elements, components, steps, and/or functions may also be added without departing from novel features disclosed herein. The apparatus, devices, and/or components illustrated in FIGS. 1-41 may be configured to perform one or more of the methods, features, or steps described herein. The novel algorithms described herein may also be efficiently implemented in software and/or embedded in hardware.

It is to be understood that the specific order or hierarchy of steps in the methods disclosed is an illustration of exemplary processes. Based upon design preferences, it is understood that the specific order or hierarchy of steps in the methods may be rearranged. The accompanying method claims present elements of the various steps in a sample order, and are not meant to be limited to the specific order or hierarchy presented unless specifically recited therein.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. As an example, “at least one of: a, b, or c” is intended to cover: a; b; c; a and b; a and c; b and c; and a, b and c. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

What is claimed is:

1. An apparatus configured for non-invasive optical spectroscopy, comprising:
a path length control part configured to control an effective optical path length of diffusely scattered light non-invasively transmitted through skin tissue of a subject to produce a target effective optical path length through the skin tissue;
a spectral sensor;
a detector configured to obtain a spectrum of an analyte of the skin tissue under test based on the diffusely scattered light; and
a light source configured to produce input light and to direct the input light towards the path length control part or the spectral sensor;
wherein the spectral sensor is configured to:
receive the input light, produce modulated light based on the input light, and direct the modulated light to the path length control part to produce the diffusely scattered light from which the spectrum is obtained by the detector, or
receive the diffusely scattered light from the path length control part and obtain the spectrum using the detector.

2. The apparatus of claim 1, further comprising:
illumination optics coupled to receive incident light corresponding to the input light or the modulated light and to direct the incident light to the skin tissue in the path length control part.

3. The apparatus of claim 2, wherein the illumination optics comprise a waveguide, a plurality of waveguides, a set of one or more lenses, or a reflector.

4. The apparatus of claim 3, wherein the reflector comprises a metallized molded part having a shape forming a compound parabolic concentrator or a compound elliptic concentrator.

5. The apparatus of claim 2, further comprising:
collection optics configured to receive the diffusely scattered light and to direct the diffusely scattered light to the spectral sensor or the detector.

6. The apparatus of claim 5, wherein the collection optics comprise a waveguide, a plurality of waveguides, a set of one or more lenses, or a reflector.

7. The apparatus of claim 6, wherein the reflector comprises a metallized molded part having a shape producing a compound parabolic concentrator or a compound elliptic concentrator.

8. The apparatus of claim 7, wherein the detector comprises a set of two or more detectors and the reflector comprises a set of two or more reflectors, each configured to direct the diffusely scattered light to a respective detector of the two or more detectors.

9. The apparatus of claim 5, wherein the illumination optics comprises a plurality of waveguides, and wherein each of the plurality of waveguides is tilted in a horizontal plane by respective angles towards an optical axis of the collection optics and each of the plurality of waveguides comprises an angle-cleaved optical fiber configured to maintain contact with the skin tissue.

10. The apparatus of claim 5, wherein the illumination optics comprises a plurality of waveguides, and wherein the plurality of waveguides are tilted in a vertical plane perpendicular to an optical axis of the diffusely scattered light transmitted through the skin tissue by a first angle and the collection optics are tilted in the vertical plane by a second angle.

11. The apparatus of claim 5, wherein the illumination optics comprises a plurality of waveguides, and further comprising:
a substrate, wherein the plurality of waveguides are integrated on the substrate.

12. The apparatus of claim 5, wherein at least one of the illumination optics or the collection optics comprises a waveguide, wherein the waveguide comprises a dielectric slab or a silicon slab.

13. The apparatus of claim 5, wherein at least one of the illumination optics or the collection optics comprises a waveguide, and further comprising:
one or more optical windows coupled to the waveguide.

14. The apparatus of claim 13, further comprising:
coupling optics configured to couple an output of the collection optics to the spectral sensor.

15. The apparatus of claim 5, wherein at least one of the illumination optics or the collection optics comprises at least one waveguide, wherein the spectral sensor comprises a micro-electro-mechanical system (MEMS) interferometer, and further comprising:
a silicon chip, wherein the at least one waveguide and the MEMS interferometer are integrated into the silicon chip.

16. The apparatus of claim 5, wherein the illumination optics and the collection optics are fixed onto a moveable tilting component configured to tilt the illumination optics and the collection optics between a first position at an angle from an optical axis of the apparatus and a second position in-plane with the optical axis of the apparatus in response to a force applied by the subject to the illumination optics and the collection optics, and wherein the path length control part comprises:
a latch configured to fix the illumination optics and the collection optics in the second position to obtain the spectrum.

17. The apparatus of claim 5, wherein the illumination optics and the collection optics are on a same axis on either side of the path length control part.

18. The apparatus of claim 5, wherein the path length control part comprises the illumination optics and the collection optics and is formed by a groove between the illumination optics and the collection optics to measure through a dermis layer of the skin tissue.

19. The apparatus of claim 1, wherein the path length control part comprises a mechanical part configured to compress and hold the skin tissue to produce the target effective optical path length.

20. The apparatus of claim 19, wherein the mechanical part comprises a pressure sensor to measure a pressure applied to the mechanical part by the subject or by the mechanical part to the skin tissue to produce pressure sensor data.

21. The apparatus of claim 20, further comprising:
a feedback device configured to adjust the mechanical part or notify a user to apply additional pressure based on at least one of the pressure sensor data or the spectrum.

22. The apparatus of claim 20, wherein the mechanical part comprises a path length measurement device configured to measure a thickness of the skin tissue corresponding to the effective optical path length, wherein the mechanical part is further configured to adjust the effective optical path length based on the thickness to produce the target effective optical path length; and
a feedback device configured to receive the thickness and adjust the effective optical path length based on the thickness.

23. The apparatus of claim 22, further comprising:
a processor configured to calculate a concentration of the analyte based on the pressure sensor data, the effective optical path length, and the spectrum.

24. The apparatus of claim 19, wherein the target effective optical path length is a fixed optical path length repeatable across respective measurements of the skin tissue.

25. The apparatus of claim 19, wherein the mechanical part is further configured to adjust the effective optical path length based on the spectrum to produce the target effective optical path length, wherein the mechanical part further comprises a feedback device configured to receive the spectrum and adjust the effective optical path length based on the spectrum.

26. The apparatus of claim 19, wherein the mechanical part further comprises at least one of illumination optics or collection optics integrated therewith.

27. The apparatus of claim 19, wherein the mechanical part comprises an opening configured to receive the skin tissue, wherein a distance between walls of the opening is configured to control the effective optical path length, and wherein pressure is applied against the mechanical part by the subject to insert the skin tissue.

28. The apparatus of claim 27, wherein the mechanical part further comprises:
a pressure sensor configured to measure the pressure applied by the subject to the mechanical part, wherein the effective optical path length is calculated based on at least one of the pressure or the spectrum.

29. The apparatus of claim 28, wherein the pressure sensor comprises a spring-loaded part.

30. The apparatus of claim 29, wherein the spring-loaded part is configured to lock into place in response to the pressure reaching a desired amount.

31. The apparatus of claim 27, wherein the opening comprises a spring-loaded moveable diffuser in a light path of the apparatus to obtain a background spectrum, wherein the pressure is applied to the spring-loaded moveable diffuser to move the skin tissue into the light path to obtain the spectrum.

32. The apparatus of claim 19, wherein the mechanical part is configured to apply at least one of mechanical pressure or suction pressure to the skin tissue, and further comprising:
illumination optics configured to direct the input light towards the skin tissue for diffused transmission of the input light through the skin tissue to produce the diffusely scattered light; and
collection optics configured to receive the diffusely scattered light from the skin tissue.

33. The apparatus of claim 32, wherein the illumination optics are configured to direct the input light towards the skin tissue at an oblique angle.

34. The apparatus of claim 1, wherein the spectral sensor comprises a spectrometer configured to receive the diffusely scattered light and to obtain the spectrum of the analyte.

35. The apparatus of claim 34, further comprising:
a non-dispersive infrared system comprising at least one narrowband light source configured to emit additional light towards the skin tissue and at least one detector configured to receive reflected light or transmitted light from the skin tissue.

36. The apparatus of claim 34, further comprising:
a laser source configured to illuminate the skin tissue at a wavelength outside an operating range of the spectrometer and corresponding to an absorption peak of the analyte.

37. The apparatus of claim 1, wherein the spectral sensor comprises an interferometer configured to receive the input light and produce the modulated light for transmission through the skin tissue to the detector.

38. The apparatus of claim 1, wherein the spectral sensor comprises a Fourier Transform infrared (FTIR) spectrometer.

39. The apparatus of claim 1, wherein the skin tissue comprises a tip of a finger, a bottom of a fingertip, an interdigital web of a hand, an earlobe, a wrist, a nose, or a portion of a neck of the subject.

40. The apparatus of claim 1, wherein the apparatus is integrated into a steering wheel of a vehicle, an ignition press button of the vehicle, a console of the vehicle, a dashboard of the vehicle, or a seatbelt of the vehicle or the apparatus is a wearable device connected to the vehicle.

41. The apparatus of claim 40, wherein the analyte of the skin tissue under test includes a blood alcohol concentration or a glucose level and wherein the apparatus controls operation of the vehicle based on the blood alcohol concentration or the glucose level.

42. The apparatus of claim 1, further comprising:
one or more ultrasonic transducers configured to excite a standing acoustic wave inside of the skin tissue to modify a refractive index of the skin tissue to reduce scattering loss inside the skin tissue.

43. The apparatus of claim 1, further comprising:
an enclosure housing the light source, the enclosure comprising an optical window for direct illumination on the skin tissue; and
free space optics configured to couple the input light to the skin tissue.

44. The apparatus of claim 43, wherein the optical window is coated with a material configured to filter a portion of the input light.

45. The apparatus of claim 1, further comprising:
an optical filter configured to filter the input light or the modulated light.
